(19) United States
(12) Patent Application Publication
(10) Pub. No.: US 2010/0021950 A1
(43) Pub. Date: Jan. 28, 2010

(54) SCREENING METHOD FOR ANTI-DIABETIC COMPOUNDS

(76) Inventors: Eckhard LAMMERT, Dresden (DE); Irena Konstantinova, Dresden (DE)

Correspondence Address: FROMMER LAWRENCE & HAUG, 745 FIFTH AVENUE- 10TH FL., NEW YORK, NY, 10151, US

(21) Appl. No.: 12/502,674

(22) Filed: Jul. 14, 2009

Related U.S. Application Data
(63) Continuation-in-part of application No. PCT/EP2008/000353, filed on Jan. 17, 2008.

(30) Foreign Application Priority Data
Jan. 17, 2007 (EP) 07000920.4

(51) Int. Cl. C12Q 1/34

(52) U.S. Cl. 435/18

(57) ABSTRACT

The present invention relates to a method of identifying a compound suitable as a lead compound and/or as a medicament for the treatment and/or prevention of diabetes, the method comprising the steps of (a) contacting a test compound with a cell comprising a protein, wherein said protein (i) comprises or consists of the amino acid sequence of any one of SEQ ID NOs: 1 to 29; (ii) is encoded by a nucleic acid molecule comprising or consisting of the sequence of any one of SEQ ID NOs: 59 to 87; (iii) is a fragment of the protein according to (i) or (ii) and exhibits Eph-Receptor-Tyrosine-Kinase activity; or (iv) has a sequence at least 75% identical with the protein according to (i) or (ii) or with the fragment according to (iii) and exhibits Eph-Receptor-Tyrosine-Kinase activity; and (b) determining whether said test compound, upon contacting in step (a) inhibits the Eph receptor tyrosine kinase activity of said protein wherein said inhibition indicates a compound suitable as a lead compound and/or as a medicament for the treatment and/or prevention of diabetes.

The present invention relates to a method of identifying a compound suitable as a lead compound and/or as a medicament for the treatment and/or prevention of diabetes, the method comprising the steps of (a) contacting a test compound with a cell comprising a protein, wherein said protein (i) comprises or consists of the amino acid sequence of any one of SEQ ID NOs: 1 to 29; (ii) is encoded by a nucleic acid molecule comprising or consisting of the sequence of any one of SEQ ID NOs: 59 to 87; (iii) is a fragment of the protein according to (i) or (ii) and exhibits Eph-Receptor-Tyrosine-Kinase activity; or (iv) has a sequence at least 75% identical with the protein according to (i) or (ii) or with the fragment according to (iii) and exhibits Eph-Receptor-Tyrosine-Kinase activity; and (b) determining whether said test compound, upon contacting in step (a) inhibits the Eph receptor tyrosine kinase activity of said protein wherein said inhibition indicates a compound suitable as a lead compound and/or as a medicament for the treatment and/or prevention of diabetes.

Throughout this specification, several documents are cited. The disclosure content of these documents is herewith incorporated by reference (including all product descriptions and manufacturers' instructions).

The treatment of type II diabetes mellitus will be a major challenge in the near future since its global prevalence has dramatically increased over the past years and is expected to increase further. Furthermore, the fact that the pathogenic basis of this disease is complex and not well understood requires multiple therapies to be employed. Owing to estimations, which expect the number of patients suffering from type II diabetes to increase from currently 190 million to over 350 million individuals worldwide within the next 15 to 20 years, there is an urgent need for effective therapeutics. This increase is supposed to be caused by a number of factors including global population growth, aging, increasing urbanization and increased obesity. The aforementioned pathophysiologic complexity renders type II diabetes mellitus a heterogeneous disease where therapeutic interventions are complex and difficult. The rise in the prevalence of obesity appears to be closely associated with an increased insulin resistance in the population. Therefore, the improvement of insulin sensitivity is thought to be a promising approach for treatment. However, besides reduced insulin sensitivity, it appears that also impairment of beta-cell function is involved in the pathogenesis of type II diabetes. It has been shown that impaired glucose regulation is associated with defects of both insulin secretion and action. In light of these discoveries, the use of therapeutic agents that are capable of improving the glucose-mediated insulin secretion is desired. Up to now, sulfonylureas have been considered to be the first-line monotherapy of choice. Also their combination with other oral anti-diabetics is suggested by many guidelines on the pharmacological treatment of diabetes worldwide although they have been discovered more than 50 years ago (Prato and Pulizzi (2006) Metabolism 55, 20). Incretins such as glucagon-like peptide-1 (GLP-1), and dipeptidyl peptidase-4 (DPP-4) inhibitors, are currently developed as alternative treatments of type II diabetes with fewer side effects (Drucker and Nauck (2006) Lancet 368, 1696).

As evident from the foregoing, proper insulin secretion is crucial to the body's ability to cope with blood glucose. Insulin is the only blood glucose-lowering hormone and is secreted by the pancreatic beta-cells of the islets of Langerhans. After its synthesis, the hormone is processed into its biologically active form and stored in vesicles ready for release. In the event insulin secretion becomes necessary, the hormone is released via a complex process involving the interplay of several proteins with the vesicle (Rorsman and Renström (2003) Diabetologia 46, 1029).

Thus, pancreatic islets are strictly required for maintaining glucose homeostasis, and defects in their ability to adequately secrete insulin in response to glucose result in diabetes mellitus (Bell and Polonsky (2001) Nature 414, 788).

During embryonic development, beta-cells initially develop as scattered single cells (Lammert et al. (2001) Science 294, 564). However, they do not stay single, but aggregate with other beta-cells and endocrine cells to form pancreatic islets. While glucose metabolism cell-autonomously triggers insulin secretion (Maechler and Wollheim (2001) 414, 807), communication between beta-cells suppresses basal insulin secretion, but enhances glucose-stimulated insulin secretion. It therefore ensures that beta-cells secrete low amounts of insulin during times of starvation, but sufficient insulin after food up-take. In comparison with intact islets, dispersed islet cells display increased basal insulin secretion and decreased glucose-stimulated insulin secretion (Halban et al. (1982) Endocrinol. 111, 86; Bosco et al. (1989) Exp. Cell Res. 184, 72; Matta et al. (1994) Pancreas 9, 439; Hopcroft et al. (1985) Endocrinol. 117, 2073). Conversely, reaggregation of islet cells suppresses basal insulin secretion and enhances glucose-stimulated insulin secretion (Halban et al. (1982) Endocrinol. 111, 86; Maes and Pipeleers (1984) Endocrinol. 114, 2205; Meda et al. (1990) J. Clin. Invest. 86, 759). Similarly, aggregation of immortalized beta-cell lines, such as mouse or rat insulinoma cells, enhances glucose-stimulated insulin secretion (Luther et al. (2006) Biochem. Biophys. Res. Commun. 343, 99). Finally, inhibition of adherent junctions and gap junctions in beta-cells provides evidence that direct cell-cell communication between beta-cells is required for physiological insulin secretion and glucose homeostasis (Dahl et al. (1996) Development 1222, 2895; Hauge-Evans et al. (1999) Diabetes 48, 1402; Yamagata et al. (2002) Diabetes 51, 114; Ravier et al. (2005) Diabetes 54, 1798). In spite of all these observations, the molecular mechanism that allows beta-cell communication to suppress basal insulin secretion, but enhance glucose-stimulated insulin secretion remains elusive.

In view of the increasing prevalence of type II diabetes mellitus in the world's population, there is an increasing need for an appropriate treatment suitable to overcome the limitations of current therapeutics. Thus, the technical problem underlying the present invention is the provision of novel means and methods useful in the treatment and/or prevention of type II diabetes mellitus.

The solution to this problem is achieved by providing the embodiments as characterized in the claims.

Accordingly, the present invention relates to a method of identifying a compound suitable as a lead compound and/or as a medicament for the treatment and/or prevention of diabetes, the method comprising the steps of (a) contacting a test compound with a cell comprising a protein, wherein said protein (i) comprises or consists of the amino acid sequence of any one of SEQ ID NOs: 1 to 29; (ii) is encoded by a nucleic acid molecule comprising or consisting of the sequence of any one of SEQ ID NOs: 59 to 87; (iii) is a fragment of the protein according to (i) or (ii) and exhibits Eph-Receptor-Tyrosine-Kinase activity; or (iv) has a sequence at least 75% identical with the protein according to (i) or (ii) or with the fragment according to (iii) and exhibits E.ph-Receptor-Tyrosine-Kinase activity; and (b) determining whether said test compound, upon contacting in step (a) inhibits the Eph receptor tyrosine kinase activity of said protein wherein said inhibition indicates a compound suitable as a lead compound and/or as a medicament for the treatment and/or prevention of diabetes.

In accordance with the present invention, the term “compound” denotes, in one alternative a small molecule. Such a small molecule may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively the compound may be an inorganic compound. Inorganic compounds are derived from mineral sources and include all compounds without carbon atoms (except carbon dioxide, carbon monoxide and carbonates). Alternatively, the compound may be a macromolecule of natural or synthetic origin. Natural macromolecules are, for example peptides, proteins such as antibodies, nucleic acid molecules such as DNA, RNA or aptamers or polysaccharides. Synthetic macromolecules are for example polymers consisting of covalently linked small organic molecules.

Small molecules have a molecular weight below 10000 Da, preferably less than 1000 Da, more preferably less than 500 Da and most preferably between 200 Da and 400 Da. Macromolecules have a molecular weight in the range of a few thousand up to several million Da. Preferably their molecular weight is more than 10000 Da, more preferably more than 100000 Da and most preferably between 150000 Da and 250000 Da.

“Nucleic acid molecules”, in accordance with the present invention, include DNA, such as cDNA or genomic DNA, and RNA. It is understood that the term “RNA” as used herein comprises all forms of RNA including mRNA, ncRNA (non-coding RNA), tRNA and rRNA. The term “non-coding RNA” includes siRNA (small interfering RNA), miRNA (micro RNA), rasiRNA (repeat associated RNA), snoRNA (small nucleolar RNA), and snRNA (small nuclear RNA). Further included are nucleic acid mimicking molecules known in the art such as synthetic or semisynthetic derivatives of DNA or RNA and mixed polymers, both sense and antisense strands. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see Braasch and Corey (2001) Chem Biol. 8, 1). LNA is an RNA derivative in which the ribose ring is constrained by a methylene linkage between the 2′-oxygen and the 4′-carbon. They may contain additional non-natural or derivatized nucleotide bases, as will be readily appreciated by those skilled in the art.

The term “antibody”, in accordance with the present invention, comprises polyclonal and monoclonal antibodies as well as derivatives or fragments thereof which still retain the binding specificity. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The antibody of the invention also includes embodiments such as chimeric, single chain and humanized antibodies, as well as antibody fragments, like, inter alia, Fab fragments, as well as fusion proteins consisting of Eph, ephrin or phosphatase extracellular domains and Fc. Antibody fragments or derivatives further comprise F(ab′)₂, Fv or scFv fragments; see, for example, Harlow and Lane (1988) and (1999), loc. cit. Various procedures are known in the art and may be used for the production of such antibodies and/or fragments. Thus, the (antibody) derivatives can be produced by peptidomimetics. Further, techniques described for the production of single chain antibodies (see, inter alia, U.S. Pat. No. 4,946,778) can be adapted to produce single chain antibodies specific for polypeptide(s) and fusion proteins of this invention. Also, transgenic animals may be used to express humanized antibodies specific for polypeptides and fusion proteins of this invention. Most preferably, the antibody of this invention is a monoclonal antibody. For the preparation of monoclonal antibodies, any technique, which provides antibodies produced by continuous cell line cultures, can be used. Examples for such techniques include the hybridoma technique (Köhler and Milstein (1975) Nature 256, 495), the trioma technique, the human B-cell hybridoma technique (Kozbor (1983) Immunology Today 4, 72) and the EBV-hybridoma technique to produce human monoclonal antibodies (Cole et al. (1985) Monoclonal Antibodies and Cancer Therapy, Alan R. Liss, Inc., 77). Surface plasmon resonance as employed in the BlAcore system can be used to increase the efficiency of phage antibodies, which bind to an epitope of a polypeptide of the invention (Schier (1996) Human Antibodies Hybridomas 7, 97; Malmborg (1995) J. Immunol. Methods 183, 7). It is also envisaged in the context of this invention that the term “antibody” comprises antibody constructs, which may be expressed in cells, e.g. antibody constructs which may be transfected and/or transduced via, amongst others, viruses or plasmid vectors. The antibody described in the context of the invention is capable to specifically bind/interact with an epitope of Eph receptor tyrosine kinases, ephrin or protein tyrosine phosphatases for Eph receptor tyrosine kinases. The term “specifically binding/interacting with” as used in accordance with the present invention means that the antibody does not or essentially does not cross-react with an epitope of similar structure. Thus, the antibody does not bind to prior art carboxylesterase of the present invention. Cross-reactivity of a panel of antibodies under investigation may be tested, for example, by assessing binding of said panel of antibodies under conventional conditions to the epitope of interest as well as to a number of more or less (structurally and/or functionally) closely related epitopes. Only those antibodies that bind to the epitope of interest in its relevant context (e.g. a specific motif in the structure of a protein) but do not or do not essentially bind to any of the other epitope are considered specific for the epitope of interest and thus to be antibodies in accordance with this invention. Corresponding methods are described e.g. in Harlow and Lane, 1988 and 1999, loc cit. The antibody specifically binds to/interacts with conformational or continuous epitopes, which are unique Eph receptor tyrosine kinases, ephrin or protein tyrosine phosphatases for Eph receptor tyrosine kinases. A conformational or discontinuous epitope is characterized for polypeptide antigens by the presence of two or more discrete amino acid residues which are separated in the primary sequence, but come together on the surface of the molecule when the polypeptide folds into the native protein/antigen (Sela (1969) Science 166, 1365; Laver (1990) Cell 61, 553). The two or more discrete amino acid residues contributing to the epitope are present on separate sections of one or more polypeptide chain(s). These residues come together on the surface of the molecule when the polypeptide chain(s) fold(s) into a three-dimensional structure to constitute the epitope. In contrast, a continuous or linear epitope consists of two or more discrete amino acid residues, which are present in a single linear segment of a polypeptide chain.

The term “aptamer” in accordance with the present invention refers to DNA or RNA molecules or peptides that have been selected from random pools based on their ability to bind other molecules. Aptamers have been selected which bind nucleic acid, proteins, small organic compounds, and even entire organisms. A database of aptamers is maintained at http://aptamer.icmb.utexas.edu/.

In addition to the above, the following definitions can define aptamers. DNA or RNA aptamers consist of (usually short) strands of oligonucleotides, whereas peptide aptamers consist of a short variable peptide domain, attached at both ends to a protein scaffold. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Peptide aptamers are proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein which have good solubility properties. Currently, the bacterial protein Thioredoxin-A is the most used scaffold protein, the variable loop being inserted within the reducing active site, which is a -Cys-Gly-Pro-Cys- loop in the wild protein, the two cysteins lateral chains being able to form a disulfide bridge. Other examples of common scaffolds include Green Fluorescent Protein (GFP), staphylococcal nuclease and the protease inhibitor Stefin A. Peptide aptamer selection can be made using different systems, but the most used is currently the yeast two-hybrid system.
Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

Non-modified aptamers are cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, etc. are available to scientists with which the half-life of aptamers easily can be increased to the day or even week time scale.

The term “lead compound” in accordance with the present invention refers to a compound discovered with step (b) of the method of the invention which will be e.g. further optimized, in particular to be pharmaceutically more acceptable. The identified lead compounds may be optimized to arrive at a compound, which may be for example used in a pharmaceutical composition. Methods for the optimization of the pharmacological properties of compounds identified in screens, the lead compounds, are known in the art and comprise a method of modifying a compound identified as a lead compound to achieve: (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmacokinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carboxylic acids, or (iii) esterification of hydroxyl groups to, e.g. phosphates, pyrophosphates or sulfates or hemi-succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiazolidines or combinations thereof.

The various steps recited above are generally known in the art. They include or rely on quantitative structure-activity relationship (QSAR) analyses (Kubinyi (1992) “Hausch-Analysis and Related Approaches”, VCH Verlag, Weinheim), combinatorial biochemistry, classical chemistry and others (see, for example, Holzgrabe and Bechtold (2000) Deutsche Apotheker Zeitung 140(8), 813).

The term “diabetes” as used throughout this specification refers to a disease in which the body does not properly control the amount of sugar in the blood. As a result, the level of sugar in the blood is too high. This disease occurs when the body does not produce enough insulin or does not use it properly. Diabetes is divided into two types, type I and type II diabetes. Type I diabetes has its onset in juvenile age and is characterized by a complete destruction of the islets of Langerhans. Type II diabetes correlates with obesity and insulin resistance, and has its onset in the adult age. It is characterized by an inability of the islets of Langerhans to secrete sufficient insulin to compensate for the body's increased demand for insulin.

The “cell” as recited throughout this specification refers to a primary cell or a cell from a cell line. Primary cells are cells which are directly obtained from an organism and which are not immortalized. Suitable primary cells are, for example, cells that are capable of secreting insulin such as pancreatic beta-cells from species such as mouse, rat, human, guinea pig, hamster, pig, dog, sheep, goat, donkey or cow, and are used either in the form of islets of Langerhans or isolated cells. The cell line may be, for example, selected from the rat insulinoma (RIN) cell lines such as INS-1 (Asfari et al. (1992) Endocrinol.130, 167), INS-2 (Asfari et al. (1992) Endocrinol.130, 167), RIN-r (Philippe et al. (1986) Endocrinol. 119, 2833) and RIN-m (Bathena et al. (1982) Diabetes 31, 521; Praz et al. (1983) Biochem. J. 210, 345; Philippe et al. (1987) J. Clin. Invest. 79, 351) or from the hamster insulinoma (HIT) cell lines such as HIT-T15 (Santerre et al. (1981) Proc. Natl. Acad. Sci. U.S.A. 78, 4339) or from mouse beta-cell lines expressing the SV40 large T-antigen (beta-TC lines) such as betaTC1, betaTC2, betaTC3 (Efrat et al. (1988) Proc. Natl. Acad. Sci. U.S.A. 85, 9037), betaTC6 (Poitout et al. (1995) Diabetes 44, 306), betaTC7 (Efrat et al. (1993) Diabetes 42, 901), betaTCtet (Efrat et al. (1995) Proc. Natl. Acad. Sci. U.S.A. 92, 3576) or from mouse insulinoma (MIN) cell lines such as MIN6 (Myazaki et al. (1990) Endocrinol. 127, 126).

As recited hereinabove, said cell comprises a protein wherein the protein comprises or consists of the amino acid sequence of any one of SEQ ID NOs: 1 to 29 or is encoded by a nucleic acid comprising or consisting of any one of SEQ ID NOs: 59 to 87. These sequences refer to the amino acid sequence or the nucleic acid sequence of different Eph receptor tyrosine kinases (see Table 1).

TABLE 1
Listing of the different amino acid and nucleic acid sequences referred to in
the present invention together with the corresponding GenBank Accession-No,
species and gene name.

| | | | SEQ ID NO | | SEQ ID NO |
|---|---|---|---|---|---|
| | | | (amino acid | | (nucleic acid |
| Name | Species | Accession No. | sequence) | Accession No. | sequence) |
| EphA1 | human | NP_005223 | 1 | NM_005232.3 | 59 |
| | mouse | NP_076069 | 2 | NM_023580.2 | 60 |
| | rat | XP_001072130 | 3 | XM_001072130 | 61 |
| EphA2 | human | NP_004422 | 4 | NM_004431.2 | 62 |
| | mouse | NP_034269 | 5 | NM_010139.2 | 63 |
| | rat | XP_001072610 | 6 | XM_001072610.1 | 64 |
| EphA4 | human | NP_004429 | 7 | NM_004438.3 | 65 |
| | mouse | NP_031962 | 8 | NM_007936.3 | 66 |
| | rat | XP_244186 | 9 | XM_244186.4 | 67 |
| EphA5 | human | NP_004430 | 10 | NM_004439.4 | 68 |
| | mouse | NP_031963 | 11 | NM_007937.2 | 69 |
| | rat | XP_001075222 | 12 | XM_001075222.1 | 70 |
| EphA7 | human | NP_004431 | 13 | NM_004440.2 | 71 |
| | mouse | NP_034271 | 14 | NM_010141.2 | 72 |
| EphA8 | human | NP_065387 | 15 | NM_020526.3 | 73 |
| | mouse | NP_031965 | 16 | NM_007939.1 | 74 |
| | rat | XP_001069777 | 17 | XM_001069777.1 | 75 |
| EphB2 | human | NP_004433 | 18 | NM_004442.6 | 76 |
| | mouse | NP_034272 | 19 | NM_010142 | 77 |
| | rat | XP_001069612 | 20 | XM_001069612.1 | 78 |
| EphB3 | human | NP_004434 | 21 | NM_004443.3 | 79 |
| | mouse | NP_034273 | 22 | NM_010143.1 | 80 |
| | rat | XP_221311 | 23 | XM_221311.4 | 81 |
| EphB4 | human | NP_004435 | 24 | NM_004444.4 | 82 |
| | mouse | NP_034274 | 25 | NM_010144.4 | 83 |
| | rat | XP_001069363 | 26 | XM_001069363.1 | 84 |
| EphB6 | human | NP_004436 | 27 | NM_004445.2 | 85 |
| | mouse | NP_031706 | 28 | NM_007680.2 | 86 |
| | rat | XP_001068490 | 29 | XM_001068490.1 | 87 |
| ephrin-A1 | mouse | NP_034237 | 30 | NM_010107.2 | 88 |
| | human | NP_004419 | 31 | NM_004428.2 | 89 |
| | rat | NP_446051 | 32 | NM_053599.2 | 90 |
| ephrin-A2 | mouse | NP_031935 | 33 | NM_007909.2 | 91 |
| | human | NP_001396 | 34 | NM_001405.2 | 92 |
| | rat | XP_001076723 | 35 | XM_001076723.1 | 93 |
| ephrin-A4 | mouse | NP_031936 | 36 | NM_007910.2 | 94 |
| | human | NP_005218 | 37 | NM_005227.2 | 95 |
| | rat | XP_001074474 | 38 | XM_001074474.1 | 96 |
| ephrin-A5 | human | NP_001953 | 39 | NM_001962.1 | 97 |
| | mouse | NP_997537 | 40 | NM_207654.1 | 98 |
| | rat | NP_446355 | 41 | NM_053903.1 | 99 |
| ephrin-B2 | mouse | NP_034241 | 42 | NM_010111.2 | 100 |
| | human | NP_004084 | 43 | NM_004093.2 | 101 |
| | rat | XP_001067856 | 44 | XM_001067856.1 | 102 |
| ephrin-B3 | human | NP_001397 | 45 | NM_001406.3 | 103 |
| | mouse | NP_031937 | 46 | NM_007911.3 | 104 |
| | rat | XP_001079433 | 47 | XM_001079433.1 | 105 |
| ACP-1d | human | NP_001035739 | 48 | NM_001040649.1 | 106 |
| ACP-1b | human | NP_009030 | 49 | NM_007099.2 | 107 |
| ACP-1c | human | NP_004291 | 50 | NM_004300.2 | 108 |
| ACP | mouse | NP_067305 | 51 | NM_021330.2 | 109 |
| ACP | rat | NP_067085 | 52 | NM_021262.2 | 110 |
| PTP-RO a | human | NP_109592 | 53 | NM_030667.1 | 111 |
| PTP-RO b | human | NP_002839 | 54 | NM_002848.2 | 112 |
| PTP-RO c | human | NP_109596 | 55 | NM_030671.1 | 113 |
| PTP-RO d | human | NP_109595 | 56 | NM_030670.1 | 114 |
| PTP-RO | mouse | NP_035346 | 57 | NM_011216.2 | 115 |
| PTP-D30 | rat | NP_059032 | 58 | NM_017336.1 | 116 |

It is of note that also all the sequences accessible through the GenBank Accession Nos. of Table 1 are within the scope of the present invention irrespective of whether the entry of the respective Accession No. is completely identical to the sequence displayed by the corresponding SEQ ID NO due to potential future updates in the database. Thus, this is to account for future corrections and modifications in the entries of GenBank, which might occur due to the continuing progress of science.

The term “fragment of the protein” as defined herein in connection with the various proteins refers to a portion of the protein comprising at least the amino acid residues necessary to maintain the biological activity of the protein.

In accordance with the invention, the term a “fragment of the protein . . . and exhibits Eph-Receptor-Tyrosine-Kinase activity” refers to a portion of the protein comprising at least the amino acid residues of the juxtamembrane region and kinase domain. Preferably, the fragment has a length of at least 200 amino acids, more preferably between 250 and 500 amino acids and most preferably between 300 and 350 amino acids. Furthermore, said fragment exhibits Eph-Receptor-Tyrosine-Kinase (Eph) activity. Eph-Receptor-Tyrosine-Kinase activity is characterized by transphosphorylation of Eph receptor intracellular domains and results in F-actin polymerization and suppression of Rac1 activity via GTP-exchange factors (GEFs) (Murai and Pasquale (2005) Neuron 46, 161). Methods to determine Eph-Receptor Tyrosine Kinase activity are well known to the person skilled in the art and include, but are not restricted to, kinase assays or assaying the binding of down-stream effectors, which are part of the Eph forward signalling pathway, to the Eph receptors. Down-stream proteins include Guanine nucleotide exchange factors (GEFs) such as ephexin (Shamah et al. (2001) Cell 105: 233) or Eph receptor tyrosine phosphatases (PTPs) such as ACP-1 (Stein et al. (1998) Genes&Dev 12:667) or PTP-RO (Shintani et al. (2006) Nat. Neurosci. 9: 761). The binding can be detected in vitro by using, for example, radioimmunoassay or ELISA.

Also encompassed by the present invention are sequences that at least exhibit 75% identity with the above-recited protein. Preferably, the identity is between 75 and 98% such as at least 80%, more preferred at least 90%, more preferred 95% and most preferred the identity is at least 98%. Such molecules may be splice forms, homologous molecules from other species, such as orthologs, or mutated sequences from the same species to mention the most prominent examples. To evaluate the identity level between two nucleotide or protein sequences, they can be aligned electronically using suitable computer programs known in the art. Such programs comprise BLAST (Altschul et al. (1990) J. Mol. Biol. 215, 403), variants thereof such as WU-BLAST (Altschul and Gish (1996) Methods Enzymol. 266, 460), FASTA (Pearson and Lipman (1988) Proc. Natl. Acad. Sci. USA 85, 2444) or implementations of the Smith-Waterman algorithm (SSEARCH, Smith and Waterman (1981) J. Mol. Biol., 147, 195). These programs, in addition to providing a pairwise sequence alignment, also report the sequence identity level (usually in percent identity) and the probability for the occurrence of the alignment by chance (P-value). Programs such as CLUSTALW (Higgins et al. (1994) Nucleic Acids Res. 22, 4673) can be used to align more than two sequences.

In accordance with the present invention an “Eph receptor tyrosine kinase” is a protein comprising extracellularly an ephrin-binding domain and two fibronectin type-IIII repeats, and a cytosolic portion comprising a juxtamembrane region, kinase domain, SAM (sterile alpha-motif) domain and a PSD-95,Deg,ZO-½ (PDZ) binding motif. These two portions are linked via a transmembrane domain. “Eph receptor tyrosine kinase activity” is, in accordance with the present invention, characterized by transphosphorylation (transfer of a phosphate group from a kinase molecule to another kinase molecule). Eph receptor tyrosine kinase activity is also characterized by interactions of Eph receptor tyrosine kinases with other proteins such as PDZ-domain-containing proteins and Rho-family guanine nucleotide exchange factors. Lastly, as discovered in the present invention, Eph receptor tyrosine kinase activity is characterized by decreased insulin secretion and Rac1 activity, as well as increased F-actin content.

“Inhibition of Eph receptor tyrosine kinase activity” is defined as a reduction of the above defined Eph receptor tyrosine kinase activity by performing one or more of the following effects: (i) the transcription of the gene encoding the protein to be inhibited is lowered, i.e. the level of mRNA is lowered, (ii) the translation of the mRNA encoding the protein to be inhibited is lowered, (iii) the protein performs its biochemical function with lowered efficiency in presence of the inhibitor, and (iv) the protein performs its cellular function with lowered efficiency in presence of the inhibitor.

In a preferred embodiment, the level of activity is less than 90%, more preferred less than 80%, 70%, 60% or 50% of the activity in absence of the inhibitor. Yet more preferred are inhibitors lowering the level down to less than 25%, less than 10%, less than 5% or less than 1% of the activity in absence of the inhibitor.

In case inhibition is the decrease of expression level of the protein, determination of the expression level of a protein can for example be carried out on the nucleic acid or protein level.

Methods for determining the expression of a protein on the nucleic acid level include, but are not limited to, northern blotting, PCR, RT-PCR or real RT-PCR. PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and terminates all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed, for example, in a 50 μl reaction mixture containing 5 μl of 10×PCR buffer with 1.5 mM MgCl₂, 200 μM of each deoxynucleoside triphosphate, 0.5 μl of each primer (10 μM), about 10 to 100 ng of template DNA and 1 to 2.5 units of Taq Polymerase. The primers for the amplification may be labeled or be unlabeled. DNA amplification can be performed, e.g., with a model 2400 thermal cycler (Applied Biosystems, Foster City, Calif.): 2 min at 94° C., followed by 30 to 40 cycles consisting of annealing (e. g. 30 s at 50° C.), extension (e. g. 1 min at 72° C., depending on the length of DNA template and the enzyme used), denaturing (e. g. 10 s at 94° C.) and a final annealing step at 55° C. for 1 min as well as a final extension step at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus Vent, Amplitaq, Pfu and KOD, some of which may exhibit proof-reading function and/or different temperature optima. However, the person skilled in the art knows how to optimize PCR conditions for the amplification of specific nucleic acid molecules with primers of different length and/or composition or to scale down or increase the volume of the reaction mix. The “reverse transcriptase polymerase chain reaction” (RT-PCR) is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerizing agents that convert the RNA target sequence into a complementary, copy-DNA (CDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746, 121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT Reaction can be performed, for example, in a 20 μl reaction mix containing: 4 μl of 5×AMV-RT buffer, 2 μl of Oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70 C.° for 15 minutes to allow for reverse transcription. The reaction temperature is then raised to 95 C.° for 1 minute to denature the RNA-cDNA duplex. Next, the reaction temperature undergoes two cycles of 95° C. for 15 seconds and 60 C.° for 20 seconds followed by 38 cycles of 90 C.° for 15 seconds and 60 C.° for 20 seconds. Finally, the reaction temperature is held at 60 C.° for 4 minutes for the final extension step, cooled to 15 C.°, and held at that temperature until further processing of the amplified sample. Any of the above mentioned reaction conditions may be scaled up according to the needs of the particular case. The resulting products are loaded onto an agarose gel and band intensities are compared after staining the nucleic acid molecules with an intercalating dye such as ethidiumbromide or SybrGreen. A lower band intensity of the sample derived from the cell treated with the test compound as compared to a non-treated cell indicates a compound that inhibits the protein.

Real-time PCR employs a specific probe, in the art also referred to as TaqMan probe, which has a reporter dye covalently attached at the 5′ end and a quencher at the 3′ end. After the TaqMan probe has been hybridized in the annealing step of the PCR reaction to the complementary site of the polynucleotide being amplified, the 5′ fluorophore is cleaved by the 5′ nuclease activity of Taq polymerase in the extension phase of the PCR reaction. This enhances the fluorescence of the 5′ donor, which was formerly quenched due to the close proximity to the 3′ acceptor in the TaqMan probe sequence. Thereby, the process of amplification can be monitored directly and in real time, which permits a significantly more precise determination of expression levels than conventional end-point PCR. Also of use in Real time RT-PCR experiments is a DNA intercalating dye such as SybrGreen for monitoring the de novo synthesis of double stranded DNA molecules.

Methods for the determination of the expression of a protein on the amino acid level include but are not limited to western blotting or polyacrylamide gel electrophoresis in conjunction with protein staining techniques such as Coomassie Brilliant blue or silver-staining. The total protein is loaded onto a polyacrylamide gel and electrophoresed. Afterwards, the separated proteins are transferred onto a membrane, e.g. a polyvinyldifluoride (PVDF) membrane, by applying an electrical current. The proteins on the membrane are exposed to an antibody specifically recognizing the protein of interest, here the Eph receptor tyrosine kinase. After washing, a second antibody specifically recognizing the first antibody and carrying a readout system such as a fluorescent dye is applied. The amount of the protein of interest is determined by comparing the fluorescence intensity of the protein derived from the cell treated with the test compound and the protein derived from a non-treated cell. A lower fluorescence intensity of the protein derived from the cell treated with the test compound indicates an inhibitor of the protein. Also of use in protein quantification is the Agilent Bioanalyzer technique.

In accordance with the present invention the term “determining whether said test compound inhibits the Eph receptor tyrosine kinase activity” refers to the determination of secreted insulin from said cell. Furthermore, it also refers to determination of Eph receptor phosphorylation, F-actin polymerization, Rac1 activity and cell-cell adhesion.

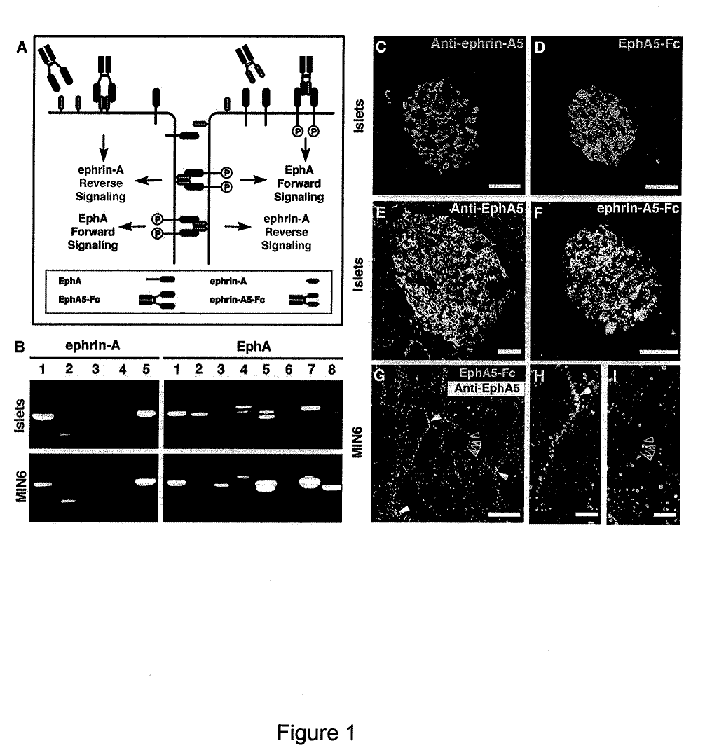

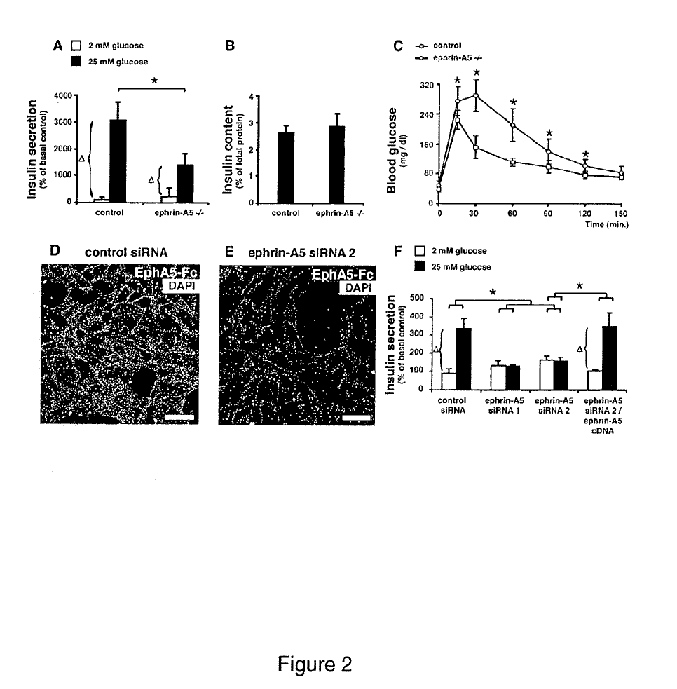

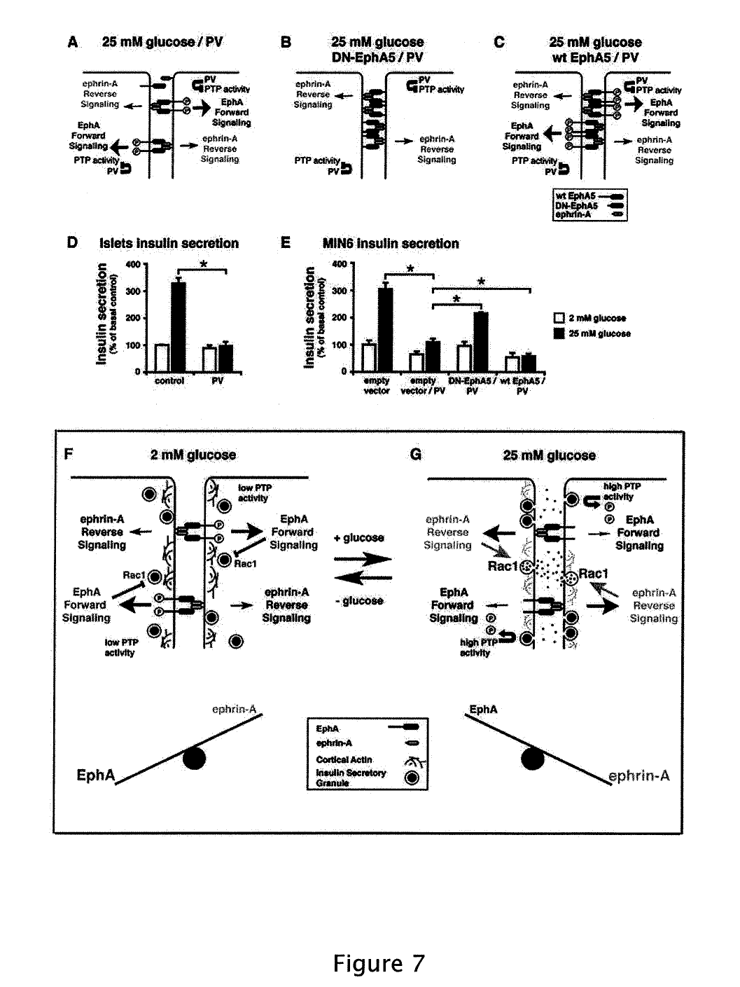

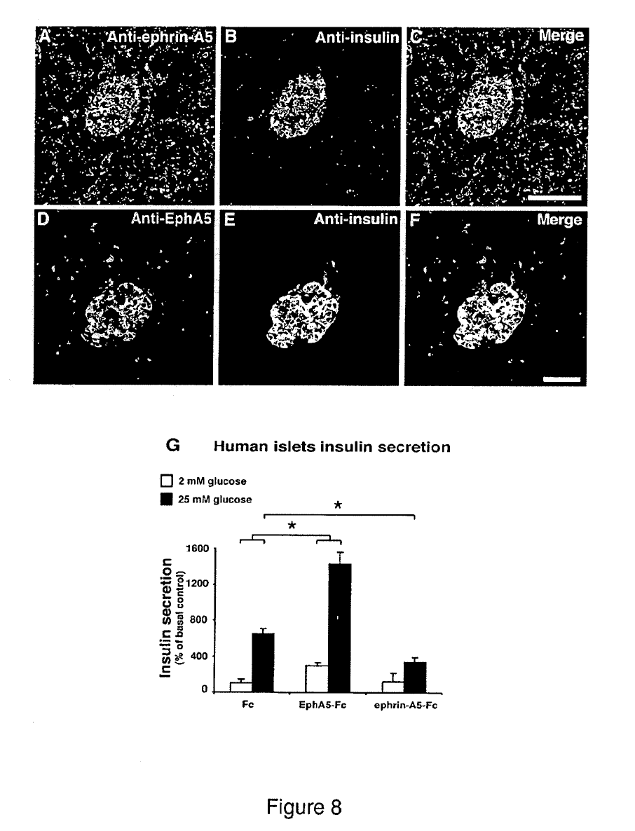

In accordance with the present invention, it was surprisingly found that, whereas the prior art provides no indication of the involvement of Eph-ephrin signalling in insulin secretion and glucose homeostasis, the interplay between Eph receptor tyrosine kinases and their corresponding ephrin ligands regulates insulin secretion in aggregated pancreatic beta-cells. The present invention for the first time shows that it is the communication between pancreatic beta-cells via Eph receptor tyrosine kinases and their ligands, the ephrins, which accounts for an optimal insulin secretory response to glucose, i.e. suppression of basal insulin secretion and enhancement of glucose-stimulated insulin secretion. Ephs and ephrins exhibit bidirectional signalling. Signalling events occur through both of the molecules. Conventionally, signalling through Ephs is referred to as forward signalling whereas signalling through ephrins is referred to as reverse signalling. Until the discoveries of the present invention, Ephs and ephrins were believed to be mainly involved in morphogenesis and pattern formation or in cell fate decisions (Pasquale (2005) Nat Rev Mol Cell Biol 6, 462). Ephs and ephrins are both present in mouse pancreatic islet cells, MIN6 cells (Example 1 and FIG. 1) as well as human pancreatic islets (Example 1 and FIG. 8). As evident from Example 2, ephrin-A5 is required for the regulation of insulin secretion from pancreatic beta-cells (see also FIG. 2). It could further be shown that Eph forward signalling, in contrast to ephrin reverse signalling, significantly decreases insulin secretion in pancreatic islets and beta-cells (see Examples 3, 5 and 7). Further evidence for the involvement of Eph in glucose stimulated insulin secretion is corroborated by phosphorylation level changes of Eph upon glucose stimulation (see Examples 9 and 10). Eph is dephosphorylated under glucose stimulation and this dephosphorylation is required for insulin secretion under stimulatory conditions. Whereas the applicants do not wish to be bound by any scientific theory, a model as outlined in FIG. 7 is proposed explaining the mechanisms underlying Eph-ephrin interactions in glucose-mediated insulin secretion. Thus, Eph receptor tyrosine kinases offer a new possibility to screen for anti-diabetic compounds as recited hereinabove.

Accordingly, the compounds identified by the above-recited method are capable of inhibiting transphosphorylation of Eph receptor tyrosine kinases. Said inhibition may be carried out, for example, by binding to the extracellular ephrin-binding site and thereby blocking said binding site without inducing the kinase activity. Inhibition may also be carried out by blocking the intracellular kinase domains of the Eph receptor tyrosine kinase and thereby preventing transphosphorylation of the receptor upon binding of the ligand. In both cases, no signalling will occur and consequently no inhibition of insulin secretion will take place. Thus, compounds that inhibit Eph receptor tyrosine kinase activity will also and in accordance with findings of the present invention increase insulin secretion. Based on the reported interaction of Eph receptors with Rho GTP-exchange factors (GEFs) (Murai and Pasquale (2005) Neuron 46, 161), an inhibition of such GEFs is also anticipated to increase insulin secretion.

To exert the above described effects, the compounds identified with the method of the present invention may act extracellularly or intracellularly. Compounds acting intracellularly may for example be an siRNA regulating down the expression level of the protein or compounds acting via inhibition of the cytosolic kinase domains. Compounds exerting their effects extracellularly may, for example, be compounds interfering with ephrin binding to the extracellular part of the Eph receptor tyrosine kinase. One example of such a compound may be an aptamer binding to the ephrin binding site.

Suitably, the method of the present invention is carried out in vitro. In vitro methods offer the possibility of establishing high-throughput assays, which are capable of screening up to several thousand compounds in parallel. High-throughput assays, independently of being biochemical, cellular or other assays, generally may be performed in wells of microtitre plates, wherein each plate may contain 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example, 10, 20, 30, 40, 50 or 100 test compounds may be added to each well. In case a well shows inhibition of Eph receptor tyrosine kinase activity, said mixture of test compounds may be de-convoluted to identify the one or more test compounds in said mixture giving rise to said inhibition.

In another embodiment, the present invention relates to a method of identifying a compound suitable as a lead compound and/or as a medicament for the treatment and/or prevention of diabetes, the method comprising the steps of (a) contacting a test compound with a protein, wherein said protein (i) comprises or consists of the amino acid sequence of any one of SEQ ID NOs: 1 to 29, or (ii) is encoded by a nucleic acid molecule comprising or consisting of the sequence of any one of SEQ ID NOs: 59 to 87, or (iii) is a fragment of the protein according to (i) or (ii) and exhibits Eph-Receptor-Tyrosine-Kinase activity, or (iv) has a sequence at least 75% identical with the protein according to (i) or (ii) or with the fragment according to (iii) and exhibits Eph-Receptor-Tyrosine-Kinase activity; and (b) determining whether said test compound, upon contacting in step (a) inhibits the Eph receptor tyrosine kinase activity of said protein wherein said inhibition indicates a compound suitable as a lead compound and/or as a medicament for the treatment and/or prevention of diabetes.

The present invention thus also provides a biochemical assay for assessing the anti-diabetic properties of a given compound. Biochemical assays offer the advantage of being carried out without the need for extensive cell culture work as compared to a cellular assay as recited above. The protein may be purified from a natural source such as tissue obtained from a mammal like mouse, rat or human. Protein purification techniques are well known to those skilled in the art. Alternatively, the protein may be recombinantly expressed in bacteria or in a cell culture. For example, nucleic acid sequences comprising a nucleic acid molecule encoding the protein can be synthesized by PCR and inserted into an expression vector (for a detailed description of the PCR process see below). Non-limiting examples of expression vectors include vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems) pTriEx-Hygro (Novagen), pCINeo (Promega), Okayama-Berg cDNA expression vector pcDVl (Pharmacia), pRc/CMV, pcDNA1, pSPORT1 (GIBCO BRL), pGEMHE (Promega), pSVL and pMSG (Pharmacia, Uppsala, Sweden), pRSVcat (ATCC 37152), pSV2dhfr (ATCC 37146) or pBC12MI (ATCC 67109). Also suitable are prokaryotic plasmid vectors such as as the pUC-series, pBluescript (Stratagene), the pET-series of expression vectors (Novagen) or pCRTOPO (Invitrogen).

After purification of the protein, said protein can be brought into contact with the test compound by combining the test compound and the protein under conditions suitable for assessing the activity of the protein. For biological assays in many cases the presence of further substances, including other salts than sodium chloride, trace elements, amino acids, vitamins, growth factors, ubiquitous co-factors such as ATP or GTP, is required. Said further substances may either be added individually or provided in complex mixtures such as serum or cell extracts. These and further accessory substances are well known in the art as are concentrations suitable for biological assays.

Conditions suitable for assessing the activity of the protein in accordance with the present invention may vary significantly, for example when comparing the interior of a cell to the extracellular space. Exemplary intracellular conditions comprise 14 mM Na⁺, 140 mM K⁺, 10⁻⁷ mM Ca²⁺, 20 mM Mg²⁺, 4 mM Cl⁻, 10 mM HCO₃⁻, 11 mM HPO₄²⁻ and H₂PO₄⁻, 1 mM SO₄²⁻, 45 mM phosphocreatine, 14 mM carnosine, 8 mM amino acids, 9 mM creatine, 1.5 mM lactate, 5 mM ATP, 3.7 mM hexose monophosphate, 4 mM protein and 4 mM urea.

After contacting the protein with the test compound, the activity of the Eph receptor tyrosine kinase is determined. To do so, a kinase assay well known to those skilled in the art may be suitable. If the test compound inhibits the kinase activity of the Eph receptor tyrosine kinase, it is suitable as a medicament or as a lead compound in accordance with the present invention. Another particularly suitable assay is the binding of down-stream effectors, which are part of the Eph forward signalling pathway, to the Eph receptors. Down-stream proteins include Guanine nucleotide exchange factors (GEFs) such as ephexin (Shamah et al. (2001) Cell 105: 233) or Eph receptor tyrosine phosphatases (PTPs) such as ACP-1 (Stein et al. (1998) Genes&Dev 12:667) or PTP-RO (Shintani et al. (2006) Nat. Neurosci. 9: 761). The binding can be detected in vitro by using radioimmunoassay or ELISA.

In another embodiment, the present invention relates to a method of identifying a compound suitable as a lead compound and/or as a medicament for the treatment and/or prevention of diabetes, the method comprising the steps of (a) contacting a test compound with a cell comprising a protein, wherein said protein (i) comprises or consists of the amino acid sequence of any one of SEQ ID NOs: 48 to 58, or (ii) is encoded by a nucleic acid molecule comprising or consisting of the sequence of any one of SEQ ID NOs: 106 to 116, or (iii) is a fragment of the protein according to (i) or (ii) and acts as a protein tyrosine phosphatase for Eph receptor tyrosine kinases; or (iv) has a sequence at least 75% identical with the protein according to (i) or (ii) or with the fragment according to (iii) and acts as a protein tyrosine phosphatase for Eph receptor tyrosine kinases; and (b) determining whether said test compound, upon contacting in step (a) activates the expression and/or activity of said protein wherein said activation indicates a compound suitable as a lead compound and/or as a medicament for the treatment and/or prevention of diabetes. As regards the definition of sequence identity in the context of the present invention, it is referred to the discussion of sequence identity above.

In accordance with the present invention the term “a protein tyrosine phosphatase for Eph receptor tyrosine kinases” refers to proteins, which dephosphorylate Eph receptor tyrosine kinases in vitro or in vivo. Examples of such protein tyrosine phosphatases (PTP) are acid phosphatase-1 (ACP-1) and its isoforms ACP-1d (protein SEQ ID NO: 48; nucleic acid sequence SEQ ID NO: 106), ACP-1b (protein SEQ ID NO: 49; nucleic acid sequence SEQ ID NO: 107), ACP-1c (protein SEQ ID NO: 50; nucleic acid sequence SEQ ID NO: 108), mouse ACP (protein SEQ ID NO: 51; nucleic acid sequence SEQ ID NO: 109), rat ACP (protein SEQ ID NO: 52; nucleic acid sequence SEQ ID NO: 110) as well as protein tyrosine phosphatase receptor type O (PTP-RO) and its isoforms PTP-RO isoform a (protein SEQ ID NO: 53; nucleic acid sequence SEQ ID NO: 111), PTP-RO isoform b (protein SEQ ID NO: 54; nucleic acid sequence SEQ ID NO: 112), PTP-RO isoform c (protein SEQ ID NO: 55; nucleic acid sequence SEQ ID NO: 113), PTP-RO isoform d (protein SEQ ID NO: 56; nucleic acid sequence SEQ ID NO: 114), mouse PTP-RO (protein SEQ ID NO: 57; nucleic acid sequence SEQ ID NO: 115) or rat receptor-type protein tyrosine phosphatase D30 (protein SEQ ID NO: 58; nucleic acid sequence SEQ ID NO: 116).

As stated above, the phosphorylation state of Eph receptor tyrosine kinases depends on the glucose concentration. Eph receptor dephosphorylation is reported to occur through acid phosphatase-1 (ACP-1, also called low molecular weight protein tyrosine phosphatase LMW-PTP) (Parri et al. (2005) J. Biol. Chem. 280, 34008) and protein tyrosine phosphatase receptor type O (PTP-RO) (Shintani et al. (2006) Nat. Neurosci. 9, 761). In accordance with the present invention, it has been discovered that glucose stimulation induces a protein tyrosine phosphatase (PTP) activity that dephosphorylates Eph in pancreatic islets and insulinoma cells (see Examples 9 and 10). Accordingly, PTPs are also useful as a target to screen for compounds modulating insulin secretion by acting via the activation of PTPs. This assay has the advantage of being performed with a cytosolic protein or protein fragment, which is often easier to handle and analyze than transmembrane proteins such as Ephs and ephrins.

Determination of the activity of said protein is accomplished, for example, by measuring the Eph receptor phosphorylation rate in cells expressing ACP-1 and/or PTP-RO. This can be done by immunoprecipitation of EphA5 from cell lysates and subsequent Western blot with a phospho-tyrosine-specific antibody (4G10) (Santa Cruz). Alternatively, Eph receptor phosphorylation is determined by using a phospho-Eph-specific antiserum (Sharma et al. (2001) Cell 105, 233). Determination of the expression level of the PTP can be carried out as described above in the context of other embodiments of the present invention.

In accordance with the invention, the term a “fragment of the protein . . . and acts as a protein tyrosine phosphatase for Eph-Receptor-Tyrosine-Kinase activity” refers to a portion of the protein that exhibits protein tyrosine phosphatase activity for Eph-Receptor-Tyrosine-Kinase. Activity of said protein has been defined above. Generally, a preferred length of the fragment in particular for PTP-RO is at least 150 amino acids, more preferably between 200 and 400 amino acids and most preferably between 220 and 250 amino acids. A preferred length of the fragment in particular for ACP is, generally, at least 100 amino acids, more preferably at least 110 amino acids and most preferably at least 112 amino acids.

In another embodiment, the invention relates to a method of identifying a compound suitable as a lead compound and/or as a medicament for the treatment and/or prevention of diabetes, the method comprising the steps of (a) contacting a test compound with a protein, wherein said protein (i) comprises or consists of the amino acid sequence of any one of SEQ ID NOs: 48 to 58, or (ii) is encoded by a nucleic acid molecule comprising or consisting of the sequence of any one of SEQ ID NOs: 106 to 116, or (iii) is a fragment of the protein according to (i) or (ii) and acts as a protein tyrosine phosphatase for Eph receptor tyrosine kinases; or (iv) has a sequence at least 75% identical with the protein according to (i) or (ii) or with the fragment according to (iii) and acts as a protein tyrosine phosphatase for Eph receptor tyrosine kinases; and (b) determining whether said test compound, upon contacting in step (a) enhances the activity of said protein wherein said enhancement indicates a compound suitable as a lead compound and/or as a medicament for the treatment and/or prevention of diabetes.

As recited above in the context of another embodiment, a biochemical assay which uses the PTP directly is also envisaged by the present invention. As outlined above, a biochemical assay offers the possibility to circumvent extensive cell culture work. A possible readout for assessing the enhancement of the activity is already provided above in the context of the corresponding cellular screening method. Detailed methods for performing all necessary steps to arrive at a suitable setup for performing the assay are also provided above in the context of the other embodiments relating to biochemical assays. A particularly suitable method is to determine the activity of the phosphatase in vitro by using a phosphopeptide of the juxtamembrane domain of Eph receptors, e.g. VDPFT(‘P’-Y)EDPN of EphA4 (Shintani et al. (2006) Nat. Neurosci. 9, 761) together with the recombinant phosphatase in a conventional protein tyrosine phosphatase assay (e.g. SIGMA). The latter method could be used for compound screens by using radioimmunoassay or ELISA.

In another embodiment, the invention relates to a method of identifying a compound suitable as a lead compound and/or as a medicament for the treatment and/or prevention of diabetes, the method comprising the steps of (a) contacting a test compound with a cell comprising a protein, wherein said protein (i) comprises or consists of the amino acid sequence of any one of SEQ ID NOs: 30 to 47, or (ii) is encoded by a nucleic acid molecule comprising or consisting of the sequence of any one of SEQ ID NOs: 88 to 105, or (iii) is a fragment of the protein according to (i) or (ii) and exhibits ephrin activity, or (iv) has a sequence at least 75% identical with the protein according to (i) or (ii) or with the fragment according to (iii) and exhibits ephrin activity; and (b) determining whether said test compound, upon contacting in step (a) activates the expression and/or activity of said protein wherein said activation indicates a compound suitable as a lead compound and/or as a medicament for the treatment and/or prevention of diabetes.

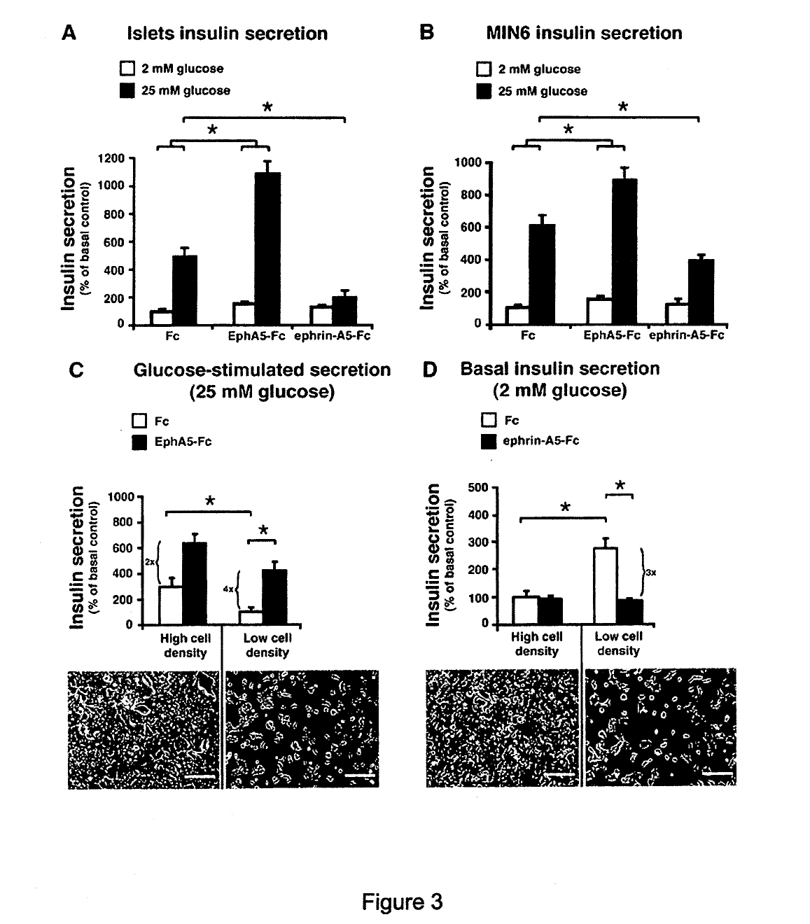

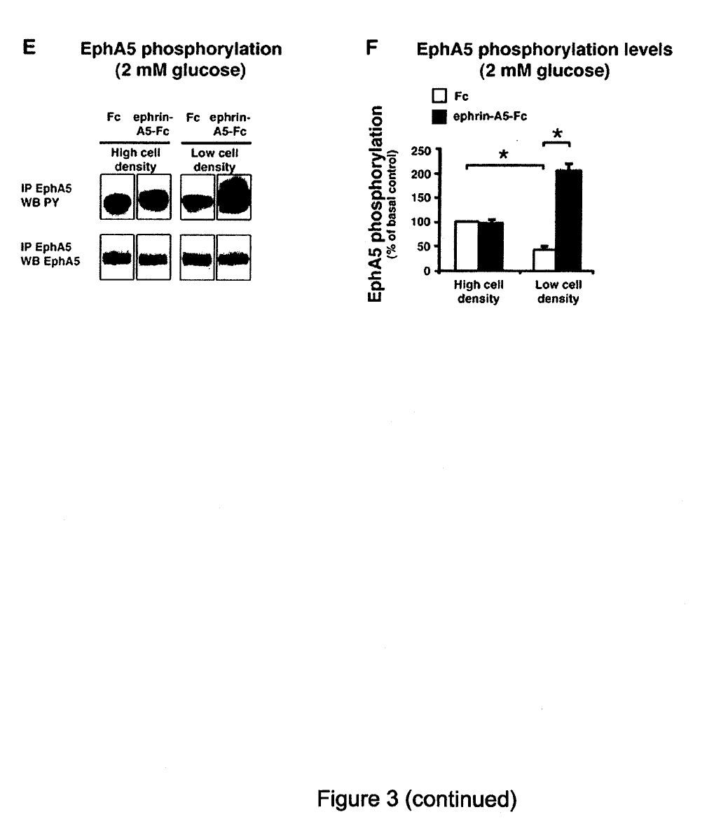

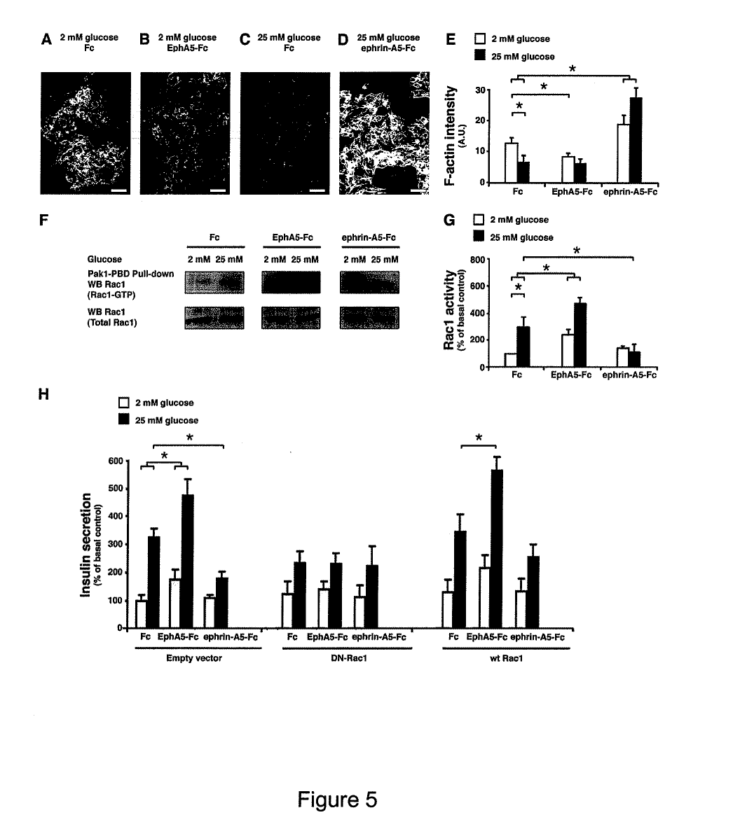

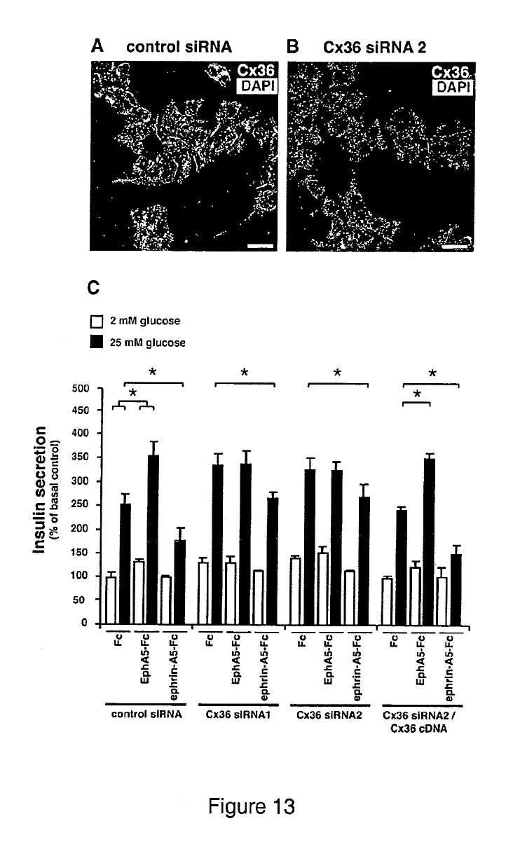

The term “ephrin activity” refers to an activity that, on the one hand induces transphosphorylation of Eph receptor tyrosine kinases and on the other hand induces ephrin reverse signalling. Ephrin reverse signalling results in enhanced activity of Rac1 as well as decreased cortical F-actin (Example 8, FIG. 5). Since ephrin-A reverse signalling is mimicked by reduced expression of the beta-cell gap junction protein connexin-36 (Example 8, FIG. 13), it is conceivable that ephrin-A reverse signalling inhibits gap junction communication between beta-cells. The main effect of ephrin-A reverse signalling is an increase in insulin secretion (Examples 3, 4 and 6, FIGS. 3, 4 and 9).

In accordance with the invention, the term a “fragment of the protein . . . and exhibits ephrin activity” refers to a portion of the protein that exhibits ephrin activity. Ephrin activity has been defined above. Generally, a preferred length of the fragment is at least 100 amino acids, more preferably at least 150 amino acids and most preferably at least 186 amino acids.

As stated above, ephrins are involved in insulin secretion from pancreatic beta-cells. Therefore, ephrins whose amino acid sequences are represented by SEQ ID NOs: 30 to 47 and whose encoding nucleic acid sequences are represented by SEQ ID NOs: 88 to 105 offer the possibility to screen for compounds in a cellular assay as described above. Ephrins represent a functionally different target as compared to Eph receptor tyrosine kinases since ephrins act in an opposite manner to Eph receptor tyrosine kinases with respect to insulin secretion (see above and Examples 3 to 7). Therefore, compounds stimulating insulin secretion via ephrins may be structurally different to compounds acting via Eph receptor tyrosine kinases.

In a preferred embodiment, the cell is a pancreatic beta-cell or a cell from a beta-cell line.

Suitable pancreatic beta-cells are, for example, from species such as mouse, rat, human, guinea pig, hamster, pig, dog, sheep, goat, donkey or cow, and are used either in the form of islets of Langerhans or isolated cells. The cell line may be, for example, selected from the rat insulinoma (RIN) cell lines such as INS-1 (Asfari et al. (1992) Endocrinol.130, 167), INS-2 (Asfari et al. (1992) Endocrinol.130, 167), RIN-r (Philippe et al. (1986) Endocrinol. 119, 2833) and RIN-m (Bathena et al. (1982) Diabetes 31, 521; Praz et al. (1983) Biochem. J. 210, 345; Philippe et al. (1987) J. Clin. Invest. 79, 351) or from the hamster insulinoma (HIT) cell lines such as HIT-T15 (Santerre et al. (1981) Proc. Natl. Acad. Sci. U.S.A. 78, 4339) or from mouse beta-cell lines expressing the SV40 large T-antigen (beta-TC lines) such as betaTC1, betaTC2, betaTC3 (Efrat et al. (1988) Proc. Natl. Acad. Sci. U.S.A. 85, 9037), betaTC6 (Poitout et al. (1995) Diabetes 44, 306), betaTC7 (Efrat et al. (1993) Diabetes 42, 901), betaTCtet (Efrat et al. (1995) Proc. Natl. Acad. Sci. U.S.A. 92, 3576) or from mouse insulinoma (MIN) cell lines such as MIN6 (Myazaki et al. (1990) Endocrinol. 127, 126).

In a preferred embodiment, determining in step (b) comprises quantifying insulin secretion.

The direct quantification of insulin secretion has the advantage of delivering a direct physiological read-out of the effect of the screened compound since insulin secretion is the desired result when screening for anti-diabetic compounds.

Preferably, determination of insulin secretion is carried out by methods including but not limited to ELISA kits (such as the one from Crystal Chem Inc.) or radioimmunoassays (such as the one from Linco, St. Charles, Mo., USA).

Generally, determination of insulin secretion is carried out at glucose concentrations in the range of 6 to 30 mM, more preferably 10 to 30 mM, and most preferably 20 to 25 mM, or in the presence of, for example, a combination of glucose and glucagon-like peptide-1 (GLP-1) or glucose and 3-isobutyl-1 -methylxanthine (IBMX).

In another preferred embodiment, determining in step (b) comprises quantifying F-actin in said cell, wherein the quantity of F-actin is determined at stimulatory glucose concentrations and wherein a decreased quantity of F-actin in the cell contacted with the test compound indicates a compound suitable as a lead compound and/or as a medicament for the treatment and/or prevention of diabetes.

The term “stimulatory glucose concentrations” generally refers to glucose concentrations in the range of 5.5 to 30 mM, more preferably 10 to 30 mM, and most preferably 20 to 25 mM. A preferred range in particular for studying activation of ephrin-A reverse signalling is 6 to 20 mM, most preferably 6 to 8 mM.

In addition to their involvement in glucose stimulated insulin secretion, the present invention shows opposite effects of Eph and ephrin signalling on F-actin. Moderate destabilization of F-actin was shown to enhance insulin secretion, while F-actin stabilization was shown to inhibit insulin secretion (Cable et al. (1995) Biochem. J. 307: 169; Wang et al. (1990) Biochem. Biophys. Res. Commun. 171: 424; Wilson et al. (2001) FEBS Lett 492: 101; Lawrence et al. (2003) Proc. Natl. Acad. Sci. U.S.A. 100: 13320; Tomas et al. (2006) J. Cell Sci. 119: 2156; Orci et al. (1972) Science 175: 1128; Jijakli et al. (2002) Int. J. Mol. Med. 9: 165; Thurmond et al. (2003) Mol. Endocrinol. 17: 732; Tsuboi et al. (2003) J. Biol. Chem. 278: 52042). The present invention now, for the first time, establishes a link between the F-actin content of a cell in response to a glucose stimulus and the molecular interaction between Ephs and ephrins (see Example 5). Quantification of F-actin is much easier than direct quantification of insulin secretion since no time-consuming assays, such as ELISA, are required but rather simple fluorescence measurements. Therefore, using the F-actin content as a readout renders the establishment of a high throughput assay for screening for anti-diabetic compounds less difficult.

Quantification of F-actin in said cell may, for example, be carried out by methods including but not limited to fluorescence measurements or F-actin ELISA.

In another preferred embodiment, the protein as referred to above is comprised in a cell expressing, or transfected with, a nucleic acid encoding said protein.

The cell is preferably a primary cell such as a pancreatic beta-cell or a cell from a cell line. Examples of suitable cell lines are referred to above.

Transfection of a cell or a cell line with a nucleic acid encoding the protein referred to above allows for adaptation of the screening method of the invention to the experimental set-up, such as the use of a different cell line capable of growing under culture conditions which are, for example, easier to implement.

In a more preferred embodiment, the cell comprising the protein referred to above is comprised in a non-human animal.

Introduction or expression of the protein referred to above in a non-human animal allows for the screening method of the invention to be performed under physiological conditions and opens the way for the identification of compounds which are, besides their anti-diabetic activity, capable of being efficiently absorbed, distributed, eventually metabolized to their biologically active form and excreted in a pharmacologically acceptable manner. Preferably, the animal is selected from, for example, rat, mouse, hamster, guinea pig, dog, cow, pig, goat, sheep or donkey.

A method for the production of a transgenic non-human animal, for example a transgenic mouse, comprises introduction of the nucleic acid molecules referred to above into a germ cell, an embryonic cell, stem cell or an egg or a cell derived therefrom. The non-human animal can be used in accordance with the invention in a method for identification of compounds useful for the treatment and/or prevention of diabetes. Production of transgenic embryos and screening of those can be performed, e.g., as described by A. L. Joyner Ed., Gene Targeting, A Practical Approach (1993), Oxford University Press. The DNA of the embryonic membranes of embryos can be analyzed using, e.g., Southern blots with an appropriate probe. A general method for making transgenic non-human animals is described in the art, see for example WO 94/24274. For making transgenic non-human organisms (which include homologously targeted non-human animals), embryonic stem cells (ES cells) are preferred. Murine ES cells, such as AB-1 line grown on mitotically inactive SNL76/7 cell feeder layers (McMahon and Bradley (1990) Cell 62,1073) essentially as described (Robertson, E. J. (1987) in Teratocarcinomas and Embryonic Stem Cells: A Practical Approach. E. J. Robertson, ed. (Oxford: IRL Press), p. 71) may be used for homologous gene targeting. Other suitable ES cell lines include, but are not limited to, the E14 line (Hooper et al. (1987) Nature 326, 292), the D3 line (Doetschman et al. (1985) J. Embryol. Exp. Morph. 87, 27), the CCE line (Robertson et al. (1986) Nature 323, 445), the AK-7 line (Zhuang et al. (1994) Cell 77, 875). The success of generating a mouse line from ES cells bearing a specific targeted mutation depends on the pluripotence of the ES cells (i. e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant non-human females and are born, e.g. as chimeric mice. The resultant transgenic mice are chimeric for cells having either the recombinase or reporter loci and are backcrossed and screened for the presence of the correctly targeted transgene(s) by PCR or Southern blot analysis on tail biopsy DNA of offspring so as to identify transgenic mice heterozygous for either the recombinase or reporter locus/loci. For example, for beta-cell specific and pancreas-specific gene manipulation, the rat insulin promoter (RIP-1 and RIP-2) and pancreas-duodenum-homeobox-1 (Pdx1) promoter can be used, respectively. These promoters are generally used to drive the gene of interest in a constitutive or inducible fashion.

In a more preferred embodiment, determining in step (b) involves quantifying insulin secretion in said non-human animal.

The invention also relates to the use of an activator of ephrin activity for the manufacture of a pharmaceutical composition for treating diabetes wherein said activator is selected from the group consisting of (a) a fusion protein consisting of the extracellular domain of an Eph receptor tyrosine kinase and an Fc chain; (b) a dominant-negative deletion mutant protein of an Eph receptor tyrosine kinase lacking the cytoplasmic tail; (c) a nucleic acid molecule encoding the protein of (a) or (b); (d) a vector comprising the nucleic acid molecule of (c); and (e) a host comprising the vector of (d).

The term “activator”, in accordance with the present invention, is defined as a compound enhancing the activity of a target molecule, preferably by performing one or more of the following effects: (i) the transcription of the gene encoding the protein to be activated is enhanced, (ii) the translation of the mRNA encoding the protein to be activated is enhanced, (iii) the protein performs its biochemical function with enhanced efficiency in presence of the activator, and (iv) the protein performs its cellular function with enhanced efficiency in presence of the activator.

In another embodiment, the activator is a small molecule. Small molecules are compounds of natural origin or chemically synthesized compounds, preferably with a molecular weight below 10000 Da, more preferred below 1000 Da, more preferred below 500 Da and most preferred between 200 Da and 400 Da.

Preferably, the small molecule may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively the compound may be an inorganic compound. Inorganic compounds are derived from mineral sources and include all compounds without carbon atoms (except carbon dioxide, carbon monoxide and carbonates).

The efficiency of the activator can be quantified by comparing the level of activity in the presence of the activator to that in the absence of the activator. For example, as an activity measure may be used: the change in amount of mRNA formed, the change in amount of protein formed, the change in amount of phosphorylation, and/or the change in the cellular phenotype or in the phenotype of an organism.

In a preferred embodiment, the level of activity is 10% more than the activity in absence of the activator, more preferred, the level of activity is 25% or 50% more than the activity in absence of the activator. Yet more preferred are activators enhancing the level of activity to 75%, 80%, 90% or 100% more than the activity in absence of the activator.

In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range 0.01 mg to 100 mg, even more preferably 0.01 mg to 50 mg and most preferably 0.01 mg to 10 mg per day.

In accordance with the present invention, “the extracellular domain of an Eph receptor tyrosine kinase” is defined as a protein comprising the extracellular domain of an Eph receptor tyrosine kinase, which generally comprises the first 200 amino acid residues of the N-terminus, preferably the first 300 amino acid residues, more preferably the first 400 amino acid residues, more preferably the first 530 amino acid residues, more preferably the first 590 amino acid residues and most preferably the first 600 amino acid residues.

The term “Fc chain” is defined as the region of an antibody composed of two heavy chains that each contribute two to three constant domains, depending on the class of the antibody. Fc chains bind to various cell receptors and complement proteins.

The “Eph receptor tyrosine kinase lacking the cytoplasmic tail” is defined as a protein which lacks the last 200-850 C-terminal amino acid residues, preferably the protein lacks the last 400-600 C-terminal amino acid residues, more preferably it lacks the last 450-550 amino acid residues and most preferably it lacks the last 470-520 C-terminal amino acid residues. Alternatively, said “Eph receptor tyrosine kinase lacking the cytoplasmic tail” may be defined as an Eph receptor tyrosine kinase comprising at least the extracellular portion and preferably said Eph receptor tyrosine kinase comprises at least the extracellular portion and the transmembrane region.

The term “dominant negative mutant” refers to a mutant that produces a protein that interacts with and/or interferes with the function of a wild-type protein.

In a preferred embodiment the nucleic acid molecule(s) is/are DNA.

The nucleic acid molecule may be inserted into several commercially available vectors. Non-limiting examples include vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems) pTriEx-Hygro (Novagen), pCINeo (Promega), Okayama-Berg cDNA expression vector pcDV1 (Pharmacia), pRc/CMV, pcDNA1, pSPORT1 (GIBCO BRL), pGEMHE (Promega), pSVL and pMSG (Pharmacia, Uppsala, Sweden), pRSVcat (ATCC 37152), pSV2dhfr (ATCC 37146) or pBC12MI (ATCC 67109).

The nucleic acid molecule referred to above may also be inserted into vectors such that a translational fusion with another nucleic acid molecule is generated. The vectors may also contain an additional expressible polynucleotide coding for one or more chaperones to facilitate correct protein folding.

For vector modification techniques, see Sambrook and Russel (2001), loc. cit. Generally, vectors can contain one or more origin of replication (ori) and inheritance systems for cloning or expression, one or more markers for selection in the host, e. g., antibiotic resistance, and one or more expression cassettes.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in eukaryotic cells are well known to those skilled in the art. These elements comprise regulatory sequences ensuring the initiation of the transcription (e. g. translation initiation codon, promoters, enhancers, and/or insulators), internal ribosomal entry sites (IRES) (Owens (2001) Proc. NatI. Acad. Sci. USA 98, 1471) and optionally poly-A signals ensuring termination of transcription and stabilization of the transcript. Additional regulatory elements may include transcriptional as well as translational enhancers, and/or naturally-associated or heterologous promoter regions. Preferably, the nucleic acid molecule is operatively linked to such expression control sequences allowing expression in eukaryotic cells. The vector may further comprise nucleotide sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Possible examples for regulatory elements ensuring the initiation of transcription comprise the cytomegalovirus (CMV) promoter, SV40-promoter, RSV-promoter (Rous sarcome virus), the lacZ promoter, the gai10 promoter, human elongation factor 1a-promoter, CMV enhancer, CaM-kinase promoter, the Autographa californica multiple nuclear polyhedrosis virus (AcMNPV) polyhedral promoter or the SV40-enhancer. Examples for further regulatory elements in prokaryotes and eukaryotic cells comprise transcription termination signals, such as SV40-poly-A site or the tk-poly-A site or the SV40, lacZ and AcMNPV polyhedral polyadenylation signals, downstream of the polynucleotide. Moreover, elements such as origin of replication, drug resistance gene, regulators (as part of an inducible promoter) may also be included. Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter).

The co-transfection with a selectable marker such as dhfr, gpt, neomycin, hygromycin allows the identification and isolation of the transfected cells. The transfected nucleic acid can also be amplified to express large amounts of the encoded (poly)peptide. The DHFR (dihydrofolate reductase) marker is useful to develop cell lines that carry several hundred or even several thousand copies of the gene of interest. Another useful selection marker is the enzyme glutamine synthase (GS) (Murphy et al. (1991) Biochem J. 227, 277; Bebbington et al. (1992) Bio/Technology 10, 169). Using these markers, the mammalian cells are grown in selective medium and the cells with the highest resistance are selected. As indicated above, the expression vectors will preferably include at least one selectable marker. Such markers include dihydrofolate reductase, G418 or neomycin resistance for eukaryotic cell culture.

The nucleic acid molecules as described hereinabove may be designed for direct introduction or for introduction via liposomes, phage vectors or viral vectors (e.g. adenoviral, retroviral) into the cell. Additionally, baculoviral systems or systems based on Vaccinia Virus or Semliki Forest Virus can be used as eukaryotic expression system for the nucleic acid molecules of the invention.

Mammalian host cells that could be used include, human Hela, HEK293, H9 and Jurkat cells, mouse NIH3T3 and C127 cells, Cos 1, Cos 7 and CV1, quail QC1-3 cells, mouse L cells and Chinese hamster ovary (CHO) cells. Furthermore, also primary cells are within the scope of the present invention. Said primary cells may be derived, for example, from mammals such as mouse, rat or human.

In a preferred embodiment, the nucleic acid molecule referred to above is comprised in a cell that is comprised in a mammal.

Preferably, said mammal is a human.

In a preferred embodiment, the pharmaceutical composition further comprises an inhibitor of an Eph receptor tyrosine kinase wherein said inhibitor is selected from the group consisting of an antisense nucleic acid molecule, a miRNA, an siRNA or an shRNA binding specifically a nucleic acid encoding said Eph receptor tyrosine kinase or from the group consisting of an antibody or aptamer.

The term “inhibitor” designates a compound lowering the activity of a target molecule, preferably by performing one or more of the following effects: (i) the transcription of the gene encoding the protein to be inhibited is lowered, (ii) the translation of the mRNA encoding the protein to be inhibited is lowered, (iii) the protein performs its biochemical function with lowered efficiency in presence of the inhibitor, and (iv) the protein performs its cellular function with lowered efficiency in presence of the inhibitor.

Compounds falling in class (i) include compounds interfering with the transcriptional machinery and/or its interaction with the promoter of said gene and/or with expression control elements remote from the promoter such as enhancers. Compounds of class (ii) comprise antisense constructs and constructs for performing RNA interference (e.g. siRNA) well known in the art (see, e.g. Zamore (2001) Nat Struct Biol. 8(9), 746; Tuschl (2001) Chembiochem. 2(4), 239). Compounds of class (iii) interfere with molecular function of the protein to be inhibited, in case of Eph receptor tyrosine kinases with its enzymatic activity, in particular with the protein kinase activity. Accordingly, active site binding compounds, in particular compounds capable of binding to the active site of any protein kinase, are envisaged. More preferred are compounds specifically binding to an active site of Eph receptor tyrosine kinases. Class (iv) includes compounds which do not necessarily bind directly to Eph receptor tyrosine kinases, but still interfere with Eph receptor tyrosine kinase activity, for example by binding to and/or inhibiting the function or inhibiting expression of members of a pathway which comprises Eph receptor tyrosine kinases. These members may be either upstream or downstream of Eph receptor tyrosine kinases within said pathway.

The efficiency of the inhibitor can be quantified by comparing the level of activity in the presence of the inhibitor to that in the absence of the inhibitor. For example, as an activity measure may be used: the change in amount of mRNA formed, the change in amount of protein formed, the change in amount of phosphorylation, and/or the change in the cellular phenotype or in the phenotype of an organism.

In a preferred embodiment, the level of activity is less than 90%, more preferred less than 80%, 70%, 60% or 50% of the activity in absence of the inhibitor. Yet more preferred are inhibitors lowering the level down to less than 25%, less than 10%, less than 5% or less than 1% of the activity in absence of the inhibitor.

The addition of an inhibitor of Eph receptor tyrosine kinases may, for example, allow for the enhancement of insulin secretion since Eph receptor tyrosine kinases inhibit insulin secretion when active (see Examples 3, 7, 9 and 10). Therefore, the therapeutic window may be, for example, wider when using a combination of ephrin activators and Eph receptor tyrosine kinase inhibitors with, for example, reduced side effects as compared to the desired effect of insulin secretion.

The present invention also relates to the use of an inhibitor of Eph receptor tyrosine kinase activity for the manufacture of a pharmaceutical composition for treating diabetes wherein said inhibitor is selected from the group consisting of (a) an antibody, aptamer, or a fragment or derivative thereof binding specifically to said Eph receptor tyrosine kinase; (b) a ribozyme specifically cleaving a nucleic acid encoding the intracellular domain of said Eph receptor tyrosine kinase; (c) a protein tyrosine phosphatase for Eph receptor tyrosine kinase; (d) a reducing agent of a protein tyrosine phosphatase for Eph receptor tyrosine kinase; (e) a nucleic acid molecule encoding the protein tyrosine phosphatase of (c); (f) a vector comprising the nucleic acid molecule of (e); (g) a host comprising the vector of (f; and (h) an inhibitor of Eph receptor tyrosine kinases which competes with ATP.

The term “Ribozymes” refers to RNA molecules that act as enzymes in the absence of proteins. These RNA molecules act catalytic or autocatalytic and are capable of cleaving e.g. other RNAs at specific target sites. Selection of appropriate target sites and corresponding ribozymes can be done as described for example in Steinecke et al. ((1995) Methods in Cell Biology 50, 449).

Examples of well-characterized small self-cleaving RNAs include, but are not restricted to, the hammerhead, hairpin, hepatitis delta virus, and in vitro-selected lead-dependent ribozymes. The organization of these small catalysts is in contrast to that of larger ribozymes, such as the group I intron.
The principle of catalytic self-cleavage has become well established in the last 10 years. The hammerhead ribozymes are characterized best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site.
The basic principle of constructing hammerhead ribozymes is as follows: An interesting region of the RNA, which contains the GUC (or CUC) triplet, is selected. Two oligonucleotide strands, each with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

Said ribozyme specifically cleaves e.g. the cytoplasmic tail of a nucleic acid encoding an Eph receptor tyrosine kinase thereby causing a truncated Eph receptor tyrosine kinase which consists, for example, only of the extracellular part and the transmembrane portion. Preferably, said ribozyme cleaves at least 500 nucleotides from 3′ end, more preferably at least 1000 nucleotides and most preferably at least 2500 from the 3′ end of the nucleic acid molecule encoding said Eph receptor tyrosine kinase.

The vector and the host are defined as recited hereinabove.

The “inhibitor of Eph receptor tyrosine kinases that competes with ATP” refers to small molecules as recited above which are additionally characterized by their ability to bind to the ATP binding site of e.g. kinases and inhibit their activity by rendering this site inaccessible for ATP. Examples include, but are not limited to, staurosporine.

Inhibition of kinase activity results in the Eph receptor tyrosine kinase's inability to exert transphosphorylation which leads to a loss of Eph receptor tyrosine kinase activity.

In a preferred embodiment, the type of diabetes to be treated is type II diabetes.

The Figures show:

FIG. 1. Expression and Localization of ephrin-As and EphAs in Pancreatic Islets and MIN6 cells

(A) Model of ephrin-A and EphA bidirectional signaling between two adjacent β-cells, and exogenous activation of ephrin-A reverse signaling and EphA forward signaling by EphA5-Fc and ephrin-A5-Fc fusion proteins, respectively. Circled P represents tyrosine phosphorylation.
(B) RT-PCR products for ephrin-A1 to -A5 and EphA1 to A8, performed with mRNA isolated from mouse islets and mouse insulinoma cells (MIN6).
(C-F) Confocal images of mouse pancreas sections show an islet surrounded by exocrine pancreatic tissue. Sections were stained with (C) Anti-ephrin-A5 antibody,
(D) EphA5-Fc that binds to ephrin-As, (E) Anti-EphA5 antibody and (F) ephrin-A5-Fc that binds to EphAs. Scale bars, 50 μm.
(G-I) Confocal images of a group of MIN6 cells stained for ephrin-As and EphA5.
(G) Low magnification, (H, I) High magnification of regions in (G): (H) Contact region between two MIN6 cells with EphA-ephrin-A colocalization (white arrowhead) and (I) free surfaces of MIN6 cells with no colocalization between ephrin-As (light grey arrowheads) and EphA5 (dark grey arrowheads). Note that the anti-EphA5 antibody was directed against the C-terminus of EphA5 and could not bind to the EphA5-Fc used to stain ephrin-As.
Scale bars in G, 10 μm, in H and I, 2 μm.

FIG. 2. Ephrin-A5 is Required for Glucose-stimulated Insulin Secretion

(A) Insulin secretion from control and ephrin-A5-/- islets at 2 mM glucose (white columns) and 25 mM glucose (black columns). Secreted insulin is normalized to insulin content and total protein content. Δ, difference between basal and glucose-stimulated insulin secretion. N=3 experiments. *p<0.05. All values are means±SD.
(B) Insulin content of islets isolated from control mice and ephrin-A5-/- mice presented as % of total protein content. N=3 experiments. *p<0.05. All values are means±SD.
(C) Glucose tolerance tests of control and ephrin-A5-/- mice. N=7 mice each. *p<0.05. All values are means±SD.
(D, E) EphA5-Fc immunostaining (white) of MIN6 cells transfected with (D) control siRNA and (E) ephrin-A5 siRNA 2. Cell nuclei are stained with DAPI (grey). Scale bars, 20 μm.
(F) Insulin secretion from MIN6 cells transfected with control siRNA, ephrin-A5 siRNA 1, ephrin-A5 siRNA 2, or co-transfected with ephrin-A5 siRNA 2 and ephrin-A5 cDNA without the targeted 3′-UTR. White columns, 2 mM glucose; black columns, 25 mM glucose. Secreted insulin is normalized to insulin content and total protein content. N=6 experiments. *p<0.05. All values are means±SD.

FIG. 3. Opposite Effects of EphA5-Fc and ephrin-A5-Fc on Insulin Secretion

(A, B) Insulin secretion from (A) mouse pancreatic islets and (B) MIN6 cells treated with Fc (as control), EphA5-Fc (to activate ephrin-A reverse signaling) and ephrin-A5-Fc (to activate EphA forward signaling) at 2 mM glucose (white columns) and 25 mM glucose (black columns), normalized to insulin content and total protein content. N=3 experiments. *p<0.05. All values are means±SD.
(C) Glucose-stimulated insulin secretion from MIN6 cells at high cell density (lower left image) and low cell density (lower right image) after treatment with Fc (white columns) or EphA5-Fc (black columns). N=4 experiments. *p<0.05. All values are means±SD. Scale bars, 100 μm.
(D) Basal insulin secretion from MIN6 cells at high cell density (lower left image) and low cell density (lower right image) after treatment with Fc (white columns) or ephrin-A5-Fc (black columns). N=3 experiments. *p<0.05. All values are means±SD. Scale bars, 100 μm.
(E) Detection of tyrosine-phosphorylated EphA5 (PY) and total EphA5 in Western blots after EphA5 immunoprecipitation from lysates of MIN6 cells that were grown at high and low cell density, and kept under basal conditions for 1 h+Fc or +ephrin-A5-Fc.
(F) EphA5 tyrosine phosphorylation, relative to total EphA5 protein, is shown in a histogram for the experiment shown in (E). N=2 experiments. *p<0.05. All values are means±SD.

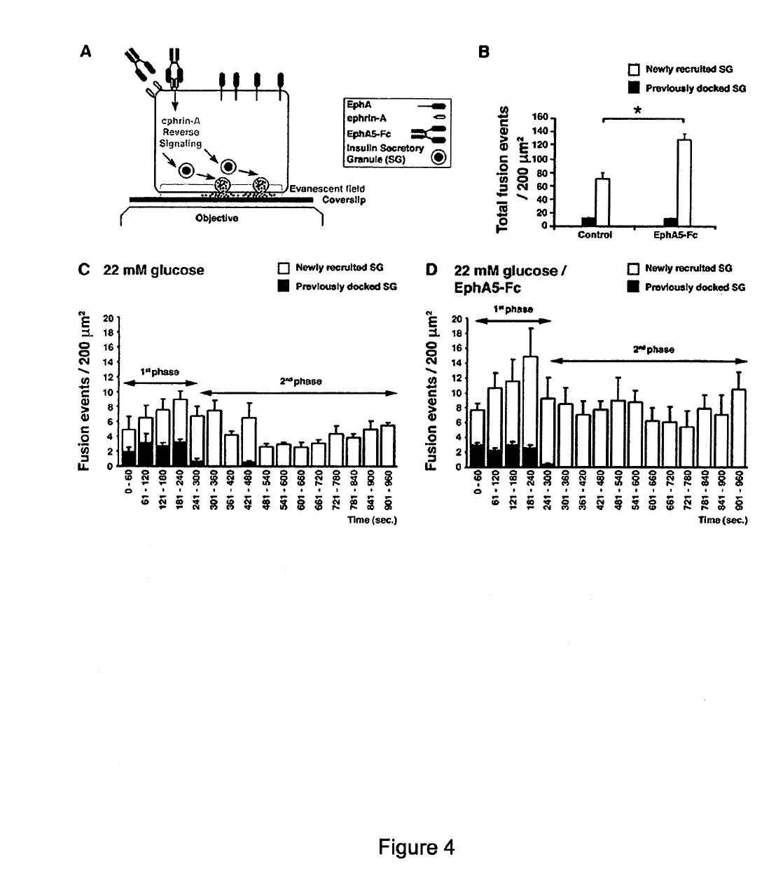

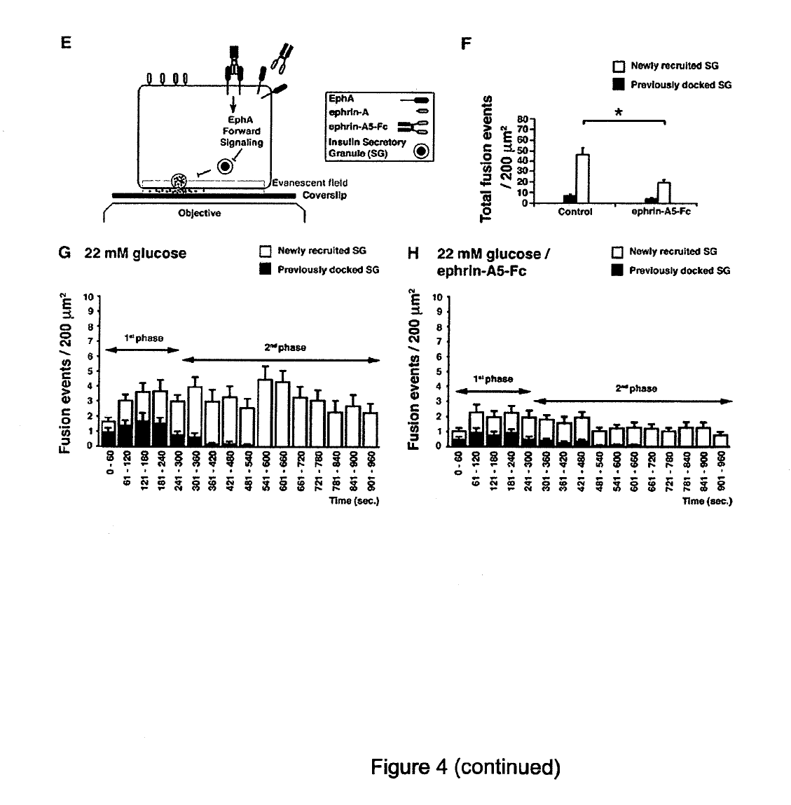

FIG. 4. Opposite Effects of ephrin-A Reverse Signaling and EphA Forward Signaling on Insulin Secretory Granule Fusion

(A) Model showing activation of ephrin-A reverse signaling by EphA5-Fc in a single insulin-GFP expressing mouse pancreatic β-cell. Total internal reflection—fluorescence microscopy (TIR-FM) was used to detect fusion of insulin secretory granules (SG) with the plasma membrane.
(B) Histogram showing all secretory fusion events in single β-cells detected during 16 min. treatment with 22 mM glucose in the absence (control) or presence of EphA5-Fc. Fusion events of previously docked SG (black columns) and newly recruited SG (white columns) were normalized to the area. N=5-7 cells each. *p<0.05. All values are means±SEM.
(C, D) Time-course of fusion events in single β-cells. Cells treated with 22 mM glucose, (C) in the absence or (D) presence of EphA5-Fc. Fusion events of newly recruited SG (white columns) and previously docked SG (black columns) are shown. N=5-7 cells each.
(E) Model showing activation of EphA forward signaling by ephrin-A5-Fc in a single insulin-GFP expressing mouse pancreatic β-cell. TIR-FM was used to detect fusion of SG with the plasma membrane.
(F) Histogram showing all secretory fusion events in single β-cells detected during 16 min. treatment with 22 mM glucose in the absence (control) or presence of ephrin-A5-Fc. Fusion events of previously docked SG (black columns) and newly recruited SG (white columns) were normalized to the area. N=20-21 cells each. *p<0.05. All values are means±SEM.
(G, H) Time-course of fusion events in single β-cells. Cells treated with 22 mM glucose, (G) in the absence or (H) presence of ephrin-A5-Fc. Fusion events of newly recruited SG (white columns) and previously docked SG (black columns) are shown. N=20-21 cells each.
FIG. 5. Opposite Effects of EphA5-Fc and ephrin-A5-Fc on F-actin and Rac1 Activity
(A-D) F-actin staining of MIN6 cells treated for 10 min. with (A) 2 mM glucose and Fc (control), (B) 2 mM glucose and EphA5-Fc (to activate ephrin-A reverse signaling), (C) 25 mM glucose and Fc (control), (D) 25 mM glucose and ephrin-A5-Fc (to activate EphA forward signaling). Scale bars, 20 μm.
(E) Quantification of F-actin fluorescence intensities in MIN6 cells treated for 10 min. with Fc, EphA5-Fc and ephrin-A5-Fc at 2 mM glucose (white columns) and 25 mM glucose (black columns). N=10 images of each N=3 coverslips were quantified. *p <0.05. All values are means±SD.
(F) Detection of active Rac1-GTP (upper bands) and total Rac1 (lower bands) in Western blots after Pakl-PBD pull-down from lysates of confluent MIN6 cells. Cells were treated for 10 min. with Fc, EphA5-Fc and ephrin-A5-Fc at 2 mM glucose (white columns) and 25 mM glucose (black columns).
(G) Corresponding histograms of Rac1 activity, relative to total Rac1 protein. N=3 experiments. *p<0.05. All values are means±SD.
(H) Insulin secretion from MIN6 cells transfected with empty vector, DN-Rac1 and wtRac1 after treatment with Fc, EphA5-Fc and ephrin-A5-Fc at 2 mM glucose (white columns) and 25 mM glucose (black columns). N=3 experiments. *p<0.05. All values are means±SD.

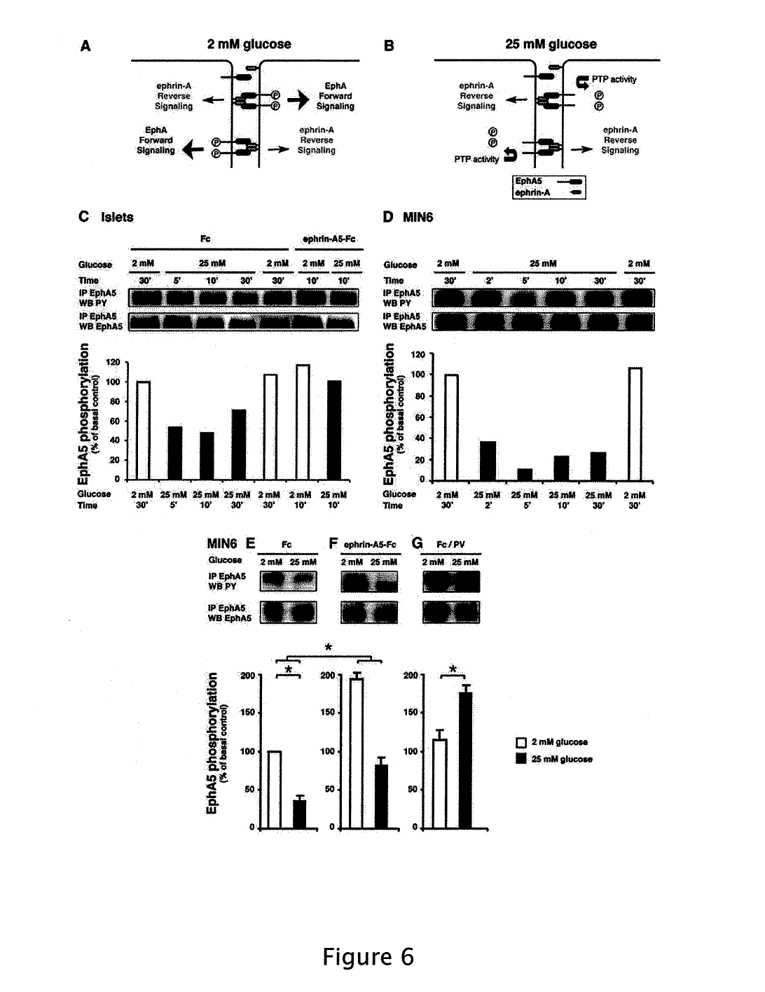

FIG. 6. Glucose-induced dephosphorylation of EphA5

(A, B) Model showing how glucose changes the outcome of EphA-ephrin-A bidirectional signaling. (A) At low glucose concentration, EphA forward signaling is active. (B) Upon glucose stimulation, EphA forward signaling is attenuated by EphA dephosphorylation, involving a protein tyrosine phosphatase (PTP) activity. (C-G) Detection of tyrosine-phosphorylated EphA5 (PY) and total EphA5 in Western blots after immunoprecipitation of EphA5 from cell lysates. The EphA5 phosphorylation level, relative to total EphA5 protein, is shown in histograms. 2 mM glucose (white columns); 25 mM glucose (black columns).
(C) Pancreatic islets treated with 2 mM glucose for 30 min., then with 25 mM glucose for 5, 10 and 30 min., followed by a switch from 25 mM to 2 mM glucose for 30 min. Islets treated with ephrin-A5-Fc (to activate EphA forward signaling) for 10 min. at 2 mM and 25 mM glucose are also shown.
(D) MIN6 cells treated with 2 mM glucose for 30 min., then with 25 mM glucose for 2, 5, 10 and 30 min., followed by a switch from 25 mM to 2 mM glucose for 30 min.
(E-G) MIN6 cells treated for 5 min. with (E) Fc (control), (F) ephrin-A5-Fc (to activate EphA forward signaling) and (G) Fc+10 μM peroxovanadate (PV) (to inhibit glucose-induced EphA5 dephosphorylation) at 2 mM and 25 mM glucose. N=2 experiments. *p<0.05. All values are means±SD.

FIG. 7. EphA Dephosphorylation is Required for Insulin Secretion

(A) Model showing that peroxovanadate (PV) blocks a PTP activity, which normally dephosphorylates EphAs at high glucose concentration. (B) Model showing that overexpression of EphA5 lacking its cytoplasmic domain (DN-EphA5) rescues inhibition of EphA forward signaling in PV-treated cells at high glucose concentration.
(C) Model showing that overexpression of wt EphA5 potentiates EphA forward signaling in PV-treated cells.
(D) Insulin secretion from mouse pancreatic islets treated with Fc in the absence or presence of 10 μM peroxovanadate (PV) at 2 mM glucose (white columns) and 25 mM glucose (black columns). Secreted insulin is normalized to insulin content and total protein content. N=3 experiments. *p<0.05. All values are means±SD.
(E) Insulin secretion from MIN6 cells transfected with an empty vector (as a control), DN-EphA5 or wt EphA5 in the absence or presence of 10 μM peroxovanadate (PV) at 2 mM glucose (white columns) and 25 mM glucose (black columns). Secreted insulin is normalized to insulin content and total protein content. N=2 experiments. *p<0.05. All values are means±SD.
(F, G) Model. (F) At low glucose concentration, interactions between EphAs and ephrin-As on adjacent β-cell plasma membranes result in EphA phosphorylation and kinase-dependent EphA forward signaling. This inhibits Rac1 activity, increases cortical F-actin and suppresses insulin secretion. (G) Upon glucose stimulation, a PTP activity dephosphorylates EphAs, and EphA -ephrin-A interactions do not lead to kinase-dependent EphA forward signaling. Instead, EphAs predominantly activate ephrin-A reverse signaling, which enhances Rac1 activity, rearranges cortical F-actin and stimulates insulin secretion.

FIG. 8. Ephrin-A5 and EphA5 Expression in the Human Pancreas: EphA5-Fc and ephrin-A5-Fc Conversely Affect Insulin Secretion from Human Pancreatic Islets

(A-F) Confocal images of human pancreas sections show an islet surrounded by exocrine pancreatic tissue. Sections are stained for (A) ephrin-A5, (B) insulin, (C) ephrin-A5 and insulin (merge), (D) EphA5, (E) insulin, (F) EphA5 and insulin (merge). Scale bars in C, 100 μm and F, 50 μm.
(G) Insulin secretion from human pancreatic islets treated with Fc (as control), EphA5-Fc (to activate ephrin-A reverse signaling) and ephrin-A5-Fc (to activate EphA forward signaling) at 2 mM glucose (white columns) and 25 mM glucose (black columns), normalized to insulin content and total protein content. N=3 experiments. *p<0.05. All values are means±SD.

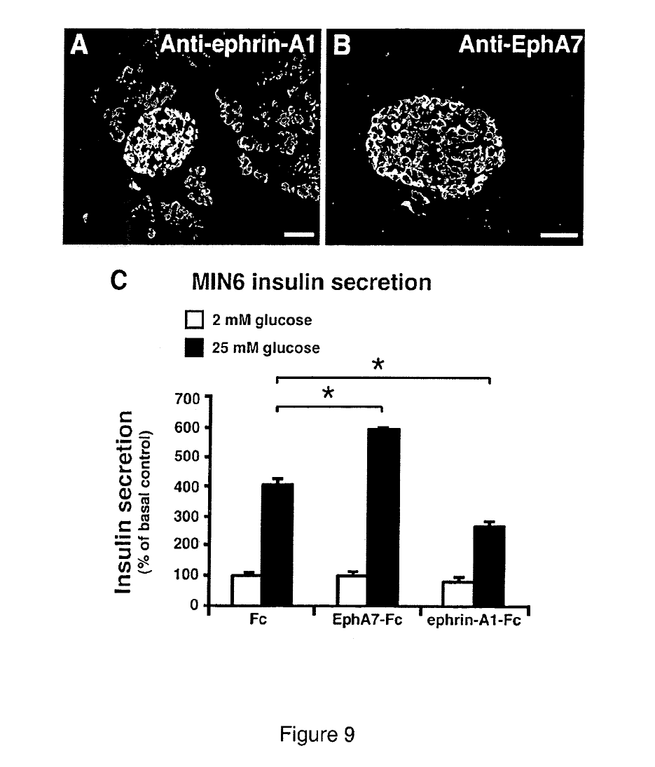

FIG. 9. Ephrin-A1 and EphA7 Expression in the Mouse Pancreas: EphA7-Fc and ephrin-A1-Fc Conversely Affect Glucose-stimulated Insulin Secretion

(A, B) Confocal images of mouse pancreas sections showing an islet surrounded by exocrine tissue. Sections were stained for (A) ephrin-A1, and (B) EphA7. Scale bars, 20 μm.
(C) Insulin secretion from MIN6 cells treated with Fc (as control), EphA7-Fc (to activate ephrin-A reverse signaling) and ephrin-A1-Fc (to activate EphA forward signaling) at 2 mM glucose (white columns) and 25 mM glucose (black columns), normalized to insulin content and total protein content. N=3 experiments. *p<0.05. All values are means±SD.

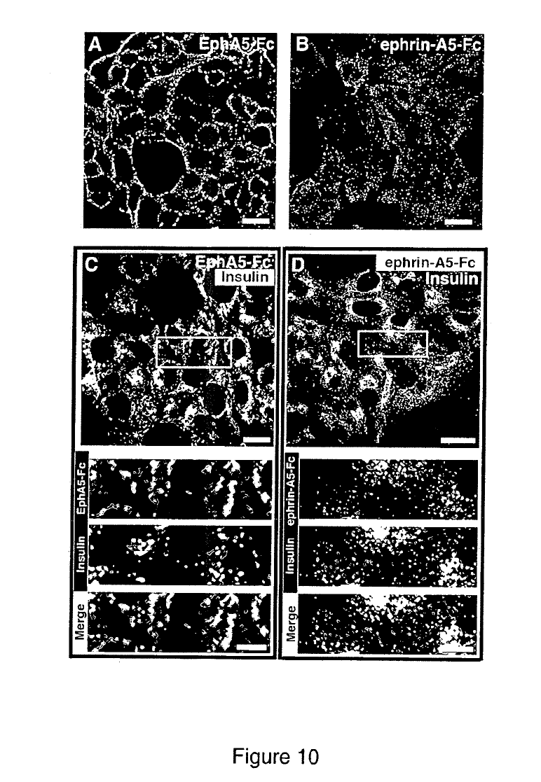

FIG. 10. Segregation of EphAs and ephrin-As in Mouse Insulinoma Cells

(A, B) Confocal images of non-permeabilized MIN6 cells stained for (A) ephrin-As with EphA5-Fc, and (B) EphAs with ephrin-A5-Fc. Scale bars, 20 μm.
(C) Confocal image of permeabilized MIN6 cells stained for ephrin-As with EphA5-Fc (white) and insulin (grey). Low magnification image, scale bar, 10 μm, and high magnification images, scale bar, 2 μm.
(D) Confocal image of permeabilized MIN6 cells stained for EphAs with ephrin-A5-Fc (grey) and insulin (white). Low magnification image, scale bar, 10 μm, and high magnification images, scale bar, 2 μm.

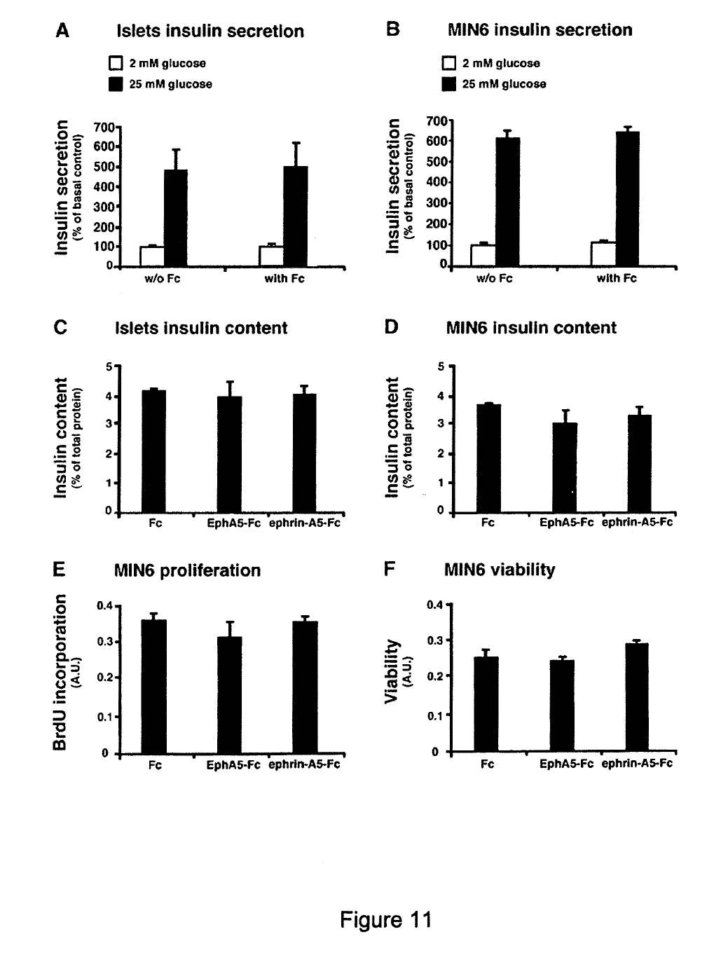

FIG. 11. Controls for Insulin Secretion Assays

(A, B) Insulin secretion from (A) mouse pancreatic islets and (B) MIN6 cells without (w/o) or with Fc control fragment at 2 mM glucose (white columns) and 25 mM glucose (black columns). Secreted insulin is normalized to insulin content and total protein content. N=3 experiments. *p<0.05. All values are means±SD.
(C, D) Insulin content of (C) mouse pancreatic islets and (D) MIN6 cells used for the insulin secretion measurements shown in FIGS. 3A and 3B, respectively. Insulin content is presented as % of total protein content. N=3 experiments. All values are means±SD.
(E) BrdU incorporation assay in MIN6 cells treated with Fc, EphA5-Fc and ephrin-A5-Fc for 1 h. N=3 experiments. All values are means±SD.
(F) WST-1 (Water-Soluble-Tetrazolium-1) viability assay of MIN6 cells treated with Fc, EphA5-Fc and ephrin-A5-Fc for 1 h. N=3 experiments. All values are means±SD.

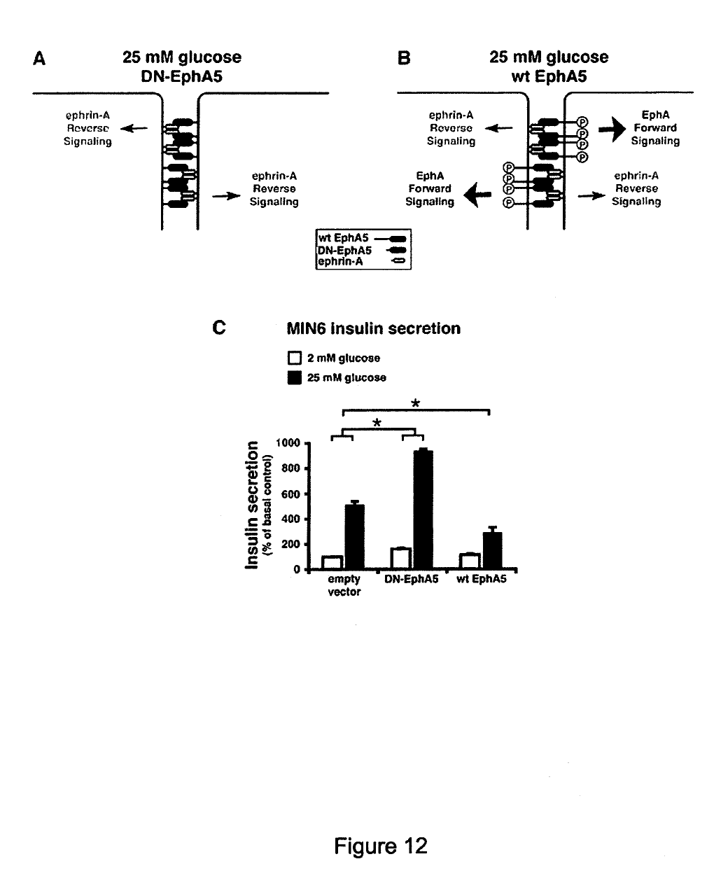

FIG. 12. EphA Forward Signaling Inhibits Insulin Secretion

(A, B) Model showing a possible role of the EphA5 cytoplasmic domain.
(A) Over-expression of dominant-negative EphA5 with no cytoplasmic domain (DN-EphA5) blocks EphA5 forward signaling and activates ephrin-A reverse signaling. (B)
Over-expression of full-length EphA5 (wt EphA5) activates EphA forward signaling and ephrin-A reverse signaling.
(C) Insulin secretion from MIN6 cells transfected with empty vector as control, DN-EphA5 and wt EphA5 at 2mM glucose (white columns) and 25 mM glucose (black columns). N=3 experiments. *p<0.05. All values are means±SD.

FIG. 13. Connexin-36 is Required for the EphA5-Fc Mediated Effect on Glucose-stimulated Insulin Secretion

(A, B) Connexin-36 (Cx36) staining (white) of MIN6 cells transfected with (A) control siRNA and (B) Cx36 siRNA 2. Cell nuclei were stained with DAPI (grey). Scale bars, 20 μm.
(C) Insulin secretion from MIN6 cells transfected with control siRNA, Cx36 siRNA 1, Cx36 siRNA 2, Cx36 siRNA2 and Cx36 cDNA at 2 mM glucose (white columns) and 25 mM glucose (black columns). N=3 experiments. *p<0.05. All values are means±SD.

The Examples illustrate the invention. In particular, the examples demonstrate the surprising finding that the interaction between Eph receptor tyrosine kinases and ephrins is the molecular basis for enhanced insulin secretion under glucose stimulation which forms the necessary scientific basis for the embodiments of the invention.

### Experimental Procedures

### Cell Culture and Transfection Procedure

MIN6 cells (Miyazaki et al. (1990) Endocrinol. 127, 126) at passages 37-47 were maintained as previously described (Nikolova et al. (2006) Dev. Cell 10, 397). The cells were electroporated with pEGFP, pEGFP-wtEphA5 (Gao et al. (1998) Proc. Natl. Acad. Sci. U.S.A. 95, 5329), pEGFP-DN-EphA5 (Gao et al. (1998) Proc. Natl. Acad. Sci. U.S.A. 95, 5329), pEGFP-ephrin-A5, (Wimmer-Kleikamp et al. (2004) J. Cell Biol. 164, 661), pEGFP-wtRac1, pEGFP-DN-Rac1 (Rac1N17) (Hall (2005) Biochem. Soc. Trans. 33, 891) and pcDNA-Cx36 constructs (Ravier et al. (2005) Diabetes 54, 1798); siRNA against firefly luciferase (control siRNA), ephrin-A5 and Cx36 by using Amaxa nucleofection (Amaxa biosystems).

### Mouse Models, Pancreatic Islet Culture and Glucose Tolerance Test

Pancreatic islets were isolated from NMRI mice for all experiments, except for the experiments with male ephrin-A5-/- and control littermates, which were C57BL/6 (Knoll et al. (2001) Development 128, 895). All mice used were 8-10 weeks old. Human pancreatic islets were isolated from a perfused human pancreas by using a protocol approved by the Ethical Research Committee of the Technical University Medical School Dresden and according to the law of the State of Saxony. Mouse and human islets were cultured overnight in Dulbecco's modified Eagle's medium (DMEM) containing 11 mM glucose (Gibco), supplemented with 10% heat inactivated fetal calf serum (FCS), 100U/ml Penicillin, 100 μg/ml Streptomycin, 1.5 g/L NaHCO₃ in a humidified atmosphere (5% CO₂, 37° C.). The glucose tolerance test was performed as previously described (Lammert et al. (2003) Curr. Biol. 13, 1070).

RT-PCR, siRNA Syntheses and Real-time RT-PCR

Total RNA was extracted from MIN6 cells and mouse pancreatic islets by using RNeasy Mini kit (Qiagen) and transcribed into cDNA, which was used for RT-PCR. All Ephs and ephrins primers were initially tested on cDNA isolated from different mouse embryonic and adult organs as positive controls.

Small interfering RNA against the 3′-UTR of ephrin-A5 and Cx36 was prepared as previously described (Nikolova et al. (2006) Dev. Cell 10, 397). Ephrin-A5 and Cx36 knockdown efficiencies were monitored at the mRNA level using real-time RT-PCR (data not shown) and immunocytochemistry.
Insulin Secretion from Pancreatic Islets and MIN6 Cells

For insulin secretion measurements, islets or MIN6 cells were starved for 1 h in Krebs Ringer Buffer (KRB) containing 115 mM NaCl, 5 mM KCl, 1.2 mM KH₂PO₄, 1 mM MgSO₄, 2.5 mM CaCl₂, 24 mM NaHCO₃, 2 mM glucose, 25 mM HEPES (pH 7.4), and 0.1% bovine serum albumin. After starvation, medium was exchanged for the same buffer +/− Fc fusion proteins to measure basal secretion, or for KRB containing 25 mM glucose +/− Fc fusion proteins to measure glucose-stimulated insulin secretion, during 1 h incubation. When Fc fusion proteins were used, islets were continuously shaken (300-500 rpm) to facilitate access of Fc fusion proteins to islet β-cells. The amount of secreted insulin was measured in the medium, and islets or MIN6 cells were subsequently dissolved in RIPA buffer to measure insulin content and total protein content. Secreted insulin was normalized to total insulin content and total protein content, and presented as % of basal control insulin secretion. In all histograms, the first column represents the basal control (=100%), except in FIG. 3C, in which the third column is taken as basal control. Secreted insulin and insulin content were measured by using ultra-sensitive rat insulin ELISA kit (Crystal Chem inc.). The total protein content was measured using BCA kit (Molecular Probes). Fc fusion proteins (R&D systems) and peroxovanadate (Sigma) were used at concentrations of 4 μg/ml and 10 μM, respectively. For determining viability and proliferation of islets and MIN6 cells during 1 h insulin secretion assay, a water-soluble tetrazolium (WST-1) reagent (Roche) and BrdU ELISA assay (Roche) were used, respectively.

### Confocal Light Microscopy and TIR-FM

Adult mouse pancreata, MIN6 cells and a biopsy of human pancreas were fixed in 4% PFA. After stepwise sucrose infiltration (9, 18, and 30%), the pancreatic tissues were embedded in O.C.T., deep frozen, and cut in 10 μm thick cryosections. 1:100-diluted rabbit-anti-EphA5, rabbit-anti-EphA7, rabbit-anti-ephrin-A1 (Santa Cruz), goat-anti-ephrin-A5 (R&D systems), guinea pig-anti-insulin (DAKO), rabbit anti-connexin-36 antibodies and 4 μg/ml Fc fusion proteins (R&D systems) were used for staining. 1:500-diluted secondary antibodies were used, conjugated with AlexaFluor488 (Molecular Probes) and with Cy5 (Dianova). 1 μg/ml DAPI (Sigma) was used to stain cell nuclei. Monolayers of MIN6 cells were treated for 10 min. with or without Fc fusion proteins at 2 mM or 25 mM glucose, fixed and stained with 1:500-diluted phalloidin-rhodamine (Molecular Probes). Confocal images were acquired by using a Zeiss confocal microscope and intensities were quantified by using ImageJ software (NIH).

The Olympus total internal reflection-fluorescence microscope (TIR-FM) was used with a high-aperture objective lens (Apo 100x OHR; NA 1.65, Olympus) (Ohara-Imaizumi et al. (2004) Biochem. J. 381, 13). To monitor single insulin granules, adenovirus (insulin-GFP) infected primary mouse β-cells on high-refractive-index glass were mounted in an open chamber and incubated for 60 min at 37° C. in KRB containing 2.2 mM glucose (starvation). Cells were pre-incubated for 15 min. +/− Fc fusion proteins under basal conditions, transferred to a thermostat-controlled stage (37° C.) and stimulated with glucose by the addition of 22 mM glucose-KRB +/− 4 μg/ml EphA5-Fc or ephrin-A5-Fc. Images were acquired every 300 ms, and images were analyzed by using Metamorph software (Molecular Devices).

### Immunoprecipitation and Western Blot

MIN6 cells and pancreatic islets were lysed in RIPA buffer, supplemented with protease and phosphatase inhibitors. 1 μg total protein, isolated from MIN6 cell lysates or 500 ng total protein, isolated from islet lysates, were used for immunoprecipitation with 1:1,000-diluted rabbit-anti-EphA5 (Santa Cruz) and protein-A beads (Amersham). For immunoprecipitation/pull-down of Rac1-GTP with Pak1-PBD (P21-activated kinase-1-P21 Binding Domain) and Westem blots, a Rac1 Activation StressXPress Kit (Biomol) was used. The immunoprecipitates were separated on 4-12% gradient SDS-polyacrylamide gels (Invitrogen) and transferred onto a PVDF membrane (Amersham). For western blots, 1:500-diluted rabbit-anti-EphA5 antibody (Santa Cruz), 1:1,000-diluted mouse-anti-PY antibody (Biomol) and 1:200-diluted rabbit-anti-Rac1 antibody (Santa Cruz) were used. 1:10,000-diluted HRP-conjugated donkey-anti-rabbit antibody (Dianova) and 1:5,000-diluted HRP-conjugated goat-anti-mouse antibody (Dianova) were used as secondary antibodies. Western blots were developed using an ECL system (Amersham). The intensities of EphA5 and Rac1 bands were normalized to the intensities of total EphA5 and Rac1, respectively, and presented as % of basal control. The intensities of the bands were quantified by using TotalLab software (Stratagene).

### Statistical Analysis

All values are means±SD. Statistical significance was determined by using the two-tailed unpaired Student's t-test, and differences were considered to be statistically significant when p<0.05.

TABLE 2

Primer sequences (5′ to 3′) used for RT-PCR and siRNA production

| SEQ. | | | |
|---|---|---|---|
| ID | | | |
| NO | type | Name | DNA |
| 117 | artificial | ephrin- | TTCAAATCCCAAGTTGCGTGAG |
| | | A1f | |
| 118 | artificial | ephrin- | AACTGTTCACCCCCTTTTCCC |
| | | A2f | |
| 119 | artificial | ephrin- | TACATCTCCACGCCCACTCACAAC |
| | | A3f | |
| 120 | artificial | ephrin- | CGATGCCTTTTGCCCCTTTC |
| | | A4f | |
| 121 | artificial | ephrin- | AACAGCAGCAACCCCAGATTC |
| | | A5f | |
| 122 | artificial | EphA1f | GCCTTACGCCAACTACACATTTACC |
| 123 | artificial | EphA2f | TGAGGATGTCCGTTTTTCCAAG |
| 124 | artificial | EphA3f | GCAATGCTGGGTATGAAGAACG |
| 125 | artificial | EphA4f | GAACAACTGGCTGCGAACTGAC |
| 126 | artificial | EphA5f | GCAAGTATTATGGGGCAGTTCG |
| 127 | artificial | EphA6f | TCCTCTTTGGTTGAAGTGCGGG |
| 128 | artificial | EphA7f | AGCAGTCTCCAGTGAACAGAATCC |
| 129 | artificial | EphA8f | TCCATCAACGAGGTAGACGAGTCC |
| 130 | artificial | ephrin- | CAGCAGCAGTGGTAGGAGCAATAC |
| | | A1r | |
| 131 | artificial | ephrin- | CTCATTGGTTGGACGCACATAAAC |
| | | A2r | |
| 132 | artificial | ephrin- | TCCCTCAAAGTCTTCCAACACG |
| | | A3r | |
| 133 | artificial | ephrin- | ATGTGATGACCCGCTCTCCTTG |
| | | A4r | |
| 134 | artificial | ephrin- | AAGCATCGCCAGGAGGAACAGTAG |
| | | A5r | |
| 135 | artificial | EphA1r | GTCCACATAGGGTTTTAGCCACAG |
| 136 | artificial | EphA2r | TGCTGTTGACGAGGATGTTGCG |
| 137 | artificial | EphA3r | TAGTTGTGATGCTGACTGCGGC |
| 138 | artificial | EphA4r | TTCAGCATTCTGCTCCTCGTGC |
| 139 | artificial | EphA5r | ATAGAGAGCAGCAGGGCAATCC |
| 140 | artificial | EphA6r | CAGTGTGTCTTGGGATGAAGCG |
| 141 | artificial | EphA7r | ATCCCAGCGGCAATACCTCTCAAC |
| 142 | artificial | EphA8r | GGGGCACTTCTTGTAGTAGATTCG |
| 143 | artificial | ephrin- | ATTAATACGACTCACTATAGGCAAGCCAGGGTTGATGAGTAG |
| | | A5 | |
| | | siRNA 1f | |
| 144 | artificial | ephrin- | ATTAATACGACTCACTATAGGAGCTGATTGCCAGGAAACAC |
| | | A5 | |
| | | siRNA 2f | |
| 145 | artificial | ephrin- | CGTAATACGACTCACTATAGGTCACAAGTTTAAATAGGACAAGCA |
| | | A5 | |
| | | siRNA 1r | |
| 146 | artificial | ephrin- | CGTAATACGACTCACTATAGGAAATGTTGGGTGCCTTCTGT |
| | | A5 | |
| | | siRNA 2r | |
| 147 | artificial | ephrin- | TGTTGACGCTGCTCTTTCTGGTG |
| | | A5f | |
| 148 | artificial | a- | CTTTGAGCCAGCCAACCAGAT |
| | | tubulinf | |
| 149 | artificial | ephrin- | AATCTGGGGTTGCTGCTGTTCC |
| | | A5r | |
| 150 | artificial | a- | TTGATGGTGGCAATGGCAG |
| | | tubulinr | |
| 151 | artificial | Cx36 | ATTAATACGACTCACTATAGGAGGGCAGGTTTGGGGAAG |
| | | siRNA 1f | |
| 152 | artificial | Cx36 | ATTAATACGACTCACTATAGGGCATGCCAGCTTTTCTTTTT |
| | | siRNA 2f | |
| 153 | artificial | Cx36 | CGTAATACGACTCACTATAGGGGCCAGATGAAGGAAAAAGA |
| | | siRNA 1r | |
| 154 | artificial | Cx36 | CGTAATACGACTCACTATAGGCGGGCTGATTAAGGTCTCTG |
| | | siRNA 2r | |
| 155 | artificial | Cx36f | ACGGTGTACGATGATGAGCA |
| 156 | artificial | Cx36r | GTAGAGTACCGGCGTTCTCG |

### EXAMPLE 1

### Ephrin-A and EphA Expression in Mouse and Human Pancreatic Islets

We investigated the presence of Ephs and ephrins in pancreatic islets as possible candidate molecules involved in cell-cell communication (FIG. 1A). First, we studied the transcriptional profiles of Ephs and ephrins in mouse islets and mouse insulinoma cells, MIN6 (FIG. 1B). We detected similar transcription of ephrin-As and EphAs in islets and MIN6 cells (FIG. 1B). We also detected expression of ephrin-Bs and EphBs (data not shown), but focused on ephrin-As and EphAs due to their strong abundance and similar expression in both islets and MIN6 cells. Immunostaining mouse pancreas sections showed co-expression of ephrin-A5 and EphA5 proteins in islets, and this expression was stronger in the islets than in the surrounding exocrine tissue (FIGS. 1C and 1E). A similar expression was observed in the human pancreas (FIG. 8A-8F). Other ephrin-As and EphAs, such as ephrin-A1 and EphA7, were also expressed in mouse pancreatic islets (FIGS. 9A and 9B). Next we stained mouse pancreas sections with EphA5-Fc and ephrin-A5-Fc fusion proteins, which bind with different affinity to virtually all ephrin-As and EphAs, respectively (FIG. 1A) (Flanagan and Vanderhaeghen (1998) Annu. Rev. Neurosci. 21, 309). The staining showed that islet ephrin-As bind to EphA5 (FIG. 1D) and that islet EphAs bind to ephrin-A5 (FIG. 1F).

EphA5 and ephrin-As co-localized in regions where MIN6 cells were in contact with each other (FIGS. 1G and 1H). In line with the findings that EphAs and ephrin-As segregate in motor neurons (Marquardt et al. (2005) Cell 121, 127), and that several transmembrane proteins also segregate in insulinoma cells (Ohara-Imaizumi et al. (2004) J. Biol. Chem. 279, 8403; Uhles et al. (2003) J. Cell Biol. 163, 1327), we observed little co-localization of EphAS and ephrin-As on the free surfaces of MIN6 cells (FIG. 11). Moreover, based on the EphA5-Fc and ephrin-A5-Fc staining of non-permeabilized MIN6 cells, we found that ephrin-As were more strongly localized to the plasma membrane compared to the EphAs (FIGS. 10A and 10B). Conversely, EphAs strongly localized to insulin secretory granules (FIG. 10D), whereas ephrin-As did not (FIG. 10C).

These results show that EphAs and ephrin-As are co-expressed in β-cells, and suggest that EphA-ephrin-A bidirectional signaling may take place between adjacent β-cells (FIG. 1A).

### EXAMPLE 2

### Ephrin-A5 is Required for Glucose-stimulated Insulin Secretion

To investigate whether ephrin-A5, a possible participant in bidirectional signaling between β-cells, was required for insulin secretion, we compared control islets with ephrin-A5 deficient islets (ephrin-A5-/-) (FIG. 2A-2C). In comparison with control islets, ephrin-A5-/- islets had a significantly reduced glucose-stimulated insulin secretion (compare black columns in FIG. 2A). These results show that ephrin-A5 is required for normal β-cell insulin secretory response to glucose (compare Δs in FIG. 2A). In contrast, we did not detect any differences in the insulin content between control and ephrin-A5-/- islets (FIG. 2B), thus excluding an effect of ephrin-A5 on insulin production. In line with the insulin secretion defects observed in ephrin-A5-/- mouse islets (FIG. 2A), we also detected impaired glucose tolerance in the ephrin-A5 deficient mice (FIG. 2C).

To rule out the possibility that the observed defects were due to changes in non-β-cells, which are also present in islets, we knocked down ephrin-A5 in MIN6 cells (FIG. 2D and 2E), a frequently used model for β-cells (Miyazaki et al. (1990) Endocrinol. 127, 126). We noticed that ephrin-A5 knockdown led to significantly increased basal insulin secretion (white columns in FIG. 2F), as well as significantly reduced glucose-stimulated insulin secretion (black columns in FIG. 2F). In addition, we could reproduce this effect with a second set of siRNA molecules. To provide evidence for the specificity of the knockdown, we rescued the insulin secretory response to glucose by cotransfecting cells with an ephrin-A5 cDNA, which lacked the 3′-UTR targeted by the siRNA (FIG. 2F). These results show that ephrin-A5 is involved in suppression of basal insulin secretion and is required for glucose-stimulated insulin secretion.

### EXAMPLE 3

### Opposite Effects of EphA5-Fc and ephrin-A5-Fc on Insulin Secretion

Communication between β-cells was shown to inhibit insulin secretion at low glucose concentrations, but stimulate insulin secretion at high glucose concentrations, and our data suggest that ephrin-A5 is involved in these communication effects. To uncover the underlying molecular mechanism, we first investigated the effects of ephrin-A reverse signaling, and EphA forward signaling, on insulin secretion (FIG. 3). We used EphA5-Fc to stimulate ephrin-A reverse signaling, and ephrin-A5-Fc to stimulate EphA forward signaling (FIGS. 3A and 3B). However, it is worth noting that EphA5-Fc and ephrin-A5-Fc fusion proteins may also interfere with endogenous EphA forward signaling and ephrin-A reverse signaling, respectively, when β-cells interact with each other.

Because ephrin-As and EphAs are promiscuous (Flanagan and Vanderhaeghen (1998) Annu. Rev. Neurosci. 21, 309), the EphA5-Fc and ephrin-A5-Fc fusion proteins allowed us to manipulate virtually all relevant ephrin-As and EphAs in β-cells, respectively. We first confirmed that control Fc fragments did not affect insulin secretion from either pancreatic islets or MIN6 cells (FIGS. 11A and 11B). We then showed that EphA5-Fc significantly increased basal and glucose-stimulated insulin secretion (compare EphA5-Fc with Fc in FIG. 3A), whereas ephrin-A5-Fc significantly decreased glucose-stimulated insulin secretion from pancreatic islets (compare ephrin-A5-Fc with Fc in FIG. 3A).

Islets consist mainly of β-cells, but also harbor other cell types, such as α-cells and endothelial cells that might have been affected in these experiments. Therefore, we tested the role of ephrin-A reverse signaling and EphA forward signaling in MIN6 cells (FIG. 3B), and obtained similar results: EphA5-Fc increased basal and glucose-stimulated insulin secretion (compare EphA5-Fc with Fc in FIG. 3B), whereas ephrin-A5-Fc decreased glucose-stimulated insulin secretion (compare ephrin-A5-Fc with Fc in FIG. 3B). Moreover, EphA7-Fc and ephrin-A1-Fc had similar effects on glucose-stimulated insulin secretion (FIG. 9C).

The effects on insulin secretion did not result from any changes in insulin content, cell division or cell viability during the 1-hour incubation time with EphA5-Fc and ephrin-A5-Fc (FIGS. 11C-11F). In addition, we also showed in human islets that EphA5-Fc strongly increased basal and glucose-stimulated insulin secretion, whereas ephrin-A5-Fc suppressed glucose-stimulated insulin secretion (FIG. 8G). These results therefore demonstrate that EphA5-Fc and ephrin-A5-Fc effectively and conversely change insulin secretion from mouse and human pancreatic islets.

### EXAMPLE 4

### EphA5-Fc Partially Rescues Glucose-stimulated Insulin Secretion in Cells with Reduced Cell-cell Contact

Glucose-stimulated insulin secretion is decreased in situations of reduced β-cell communication. Therefore we asked whether EphA5-Fc restored glucose-stimulated insulin secretion in non-confluent MIN6 cells (FIG. 3C). We first showed that non-confluent cells (“Low cell density”) had lower glucose-stimulated insulin secretion compared to confluent cells (“High cell density”) (compare white columns in FIG. 3C). We then showed that EphA5-Fc partially restored glucose-stimulated insulin secretion in non-confluent cells (compare last two columns in FIG. 3C). These results suggest that endogenous ephrin-A reverse signaling enhances glucose-stimulated insulin secretion.

### EXAMPLE 5

### Ephrin-A5-Fc Fully Rescues Suppression of Basal Insulin Secretion in Cells with Reduced Cell-cell Contact

In the experiments with islets, as well as confluent MIN6 cell monolayers, we noticed that ephrin-A5-Fc reduced glucose-stimulated insulin secretion, but did not suppress basal insulin secretion (FIGS. 3A and 3B). We hypothesized that basal insulin secretion already was maximally suppressed by endogenous EphA-ephrin-A interactions and, therefore, we could not further decrease basal insulin secretion by using ephrin-A5-Fc. If this were the case, one would expect that ephrin-A5-Fc was capable of decreasing basal insulin secretion, when the extent of endogenous EphA-ephrin-A interactions was less. Thus, we performed the experiments with non-confluent MIN6 cells; which had lower levels of endogenous EphA forward signaling as a result of less cell-cell contact.

As shown in FIG. 3D, non-confluent MIN6 cells had significantly enhanced basal insulin secretion (compare white columns). In support of the hypothesis that this was due to reduced endogenous EphA forward signaling, we observed that EphA5 phosphorylation levels were lower in non-confluent MIN6 cells compared to confluent cells (FIG. 3E; compare white columns in FIG. 3F). Importantly, ephrin-A5-Fc decreased basal insulin secretion in non-confluent cells to a level characteristic of confluent MIN6 cells (compare last two columns in FIG. 3D). Consistent with the idea that this was due to exogenous activation of EphA forward signaling, EphA5 phosphorylation levels were significantly increased upon ephrin-A5-Fc treatment of non-confluent MIN6 cells (FIG. 3E; compare last two columns in FIG. 3F). Ephrin-A5-Fc treatment even induced a stronger EphA5 phosphorylation in non-confluent MIN6 cells than in confluent ones (FIG. 3F), possibly due to increased fusion of insulin secretory granules, which carry EphAs to the plasma membrane, where they can bind to ephrin-A5-Fc. These results suggest that endogenous EphA forward signaling inhibits basal insulin secretion.

### EXAMPLE 6

### Ephrin-A Reverse Signaling Stimulates Insulin Secretion

The previous experiments strongly suggested that ephrin-A reverse signaling enhanced insulin secretion (FIG. 3). Here we used single primary β-cells to stimulate ephrin-A reverse signaling with EphA5-Fc, without simultaneously affecting endogenous EphA forward signaling (FIG. 4A). This was possible because the cells did not contact each other and, therefore, their ephrin-As could not interact with the EphAs of any adjacent cells (FIG. 4A).

We expressed GFP-tagged insulin in primary mouse β-cells and stimulated these cells with glucose, in the presence or absence of EphA5-Fc (FIG. 4A). We then monitored the secretory events in single β-cells by using total internal reflection-fluorescence microscopy (TIR-FM). Time-lapse TIR-FM allowed us to discriminate between fusion events of previously docked and newly recruited insulin secretory granules (Ohara-Imaizumi et al. (2004) Biochem. J. 381, 13). As shown in FIG. 4B, EphA5-Fc treatment significantly increased fusion events of newly recruited insulin secretory granules (white columns).

Glucose-stimulated insulin secretion, in response to a sudden increase in glucose concentration, follows a biphasic time course consisting of a rapid 1^{st} phase followed by a sustained 2^{nd} phase (Rorsman and Renstrom (2003) Diabetologia 46, 1029). We found that EphA5-Fc increased the number of secretory events during both phases of insulin secretion with a stronger effect on the 2^{nd} phase (compare FIGS. 4C and 4D). We conclude from these experiments that ephrin-A reverse signaling stimulates insulin secretion.

### EXAMPLE 7

### EphA Forward Signaling Suppresses Insulin Secretion

The experiments, shown in FIG. 3, strongly suggested that EphA forward signaling suppressed insulin secretion. Here we used TIR-FM of single ephrin-A5-Fc treated β-cells to provide evidence of a suppressive effect of EphA forward signaling (FIG. 4E). As shown in FIG. 4F, ephrin-A5-Fc treatment significantly reduced the number of fusion events of newly recruited insulin secretory granules (white columns). In addition, ephrin-A5-Fc reduced the number of secretory events during both phases of insulin secretion with a stronger effect on the 2^{nd} phase (compare FIGS. 4G and 4H). As additional evidence of an inhibitory effect of EphA forward signaling on insulin secretion, a dominant-negative EphA5 protein (DN-EphA5), lacking its cytoplasmic domain, was expressed in MIN6 cells (FIG. 12A). This DN-EphA5 binds to ephrin-As and this binding is expected to induce ephrin-A reverse signaling, but not EphA forward signaling. In contrast, over-expression of a full-length EphA5 protein (wt EphA5) is expected to induce ephrin-A reverse signaling, and also EphA forward signaling (FIG. 12B). In support of an inhibitory effect of EphA forward signaling, we found that DN-EphA5 increased basal and glucose-stimulated insulin secretion, whereas wt EphA5 over-expression decreased glucose-stimulated insulin secretion in confluent MIN6 cell monolayers (FIG. 12C). We conclude that EphA forward signaling suppresses insulin secretion.

### EXAMPLE 8

### Downstream Targets of EphA-ephrin-A Signaling and Their Impact on Insulin Secretion

It has been shown that destabilization of F-actin enhances insulin secretion, whereas its stabilization inhibits insulin secretion (Tomas et al. (2006) J. Cell Sci. 119, 2156). This led to the hypothesis that dense cortical F-actin network limits access of insulin secretory granules to the plasma membrane. However, since complete F-actin depolymerization inhibits glucose-stimulated insulin secretion (Li et al. (1994) Mol. Biol. Cell 5, 1199), certain actin filaments appear to be required for the secretory process.

Because ephrin-A reverse signaling, and EphA forward signaling, were shown to rearrange F-actin in several cell types (Marquardt et al. (2005) Cell 121, 127), we tested whether EphA5-Fc and ephrin-A5-Fc also changed the F-actin state of mouse insulinoma cells (FIG. 5A-5E). We found that EphA5-Fc resulted in F-actin rearrangements with a subtle decrease in F-actin intensity under basal conditions (compare FIGS. 5A and 5B), consistent with its stimulatory effect on insulin secretion. In contrast, ephrin-A5-Fc strongly increased F-actin polymerization under basal and stimulatory conditions (compare FIGS. 5C and 5D), consistent with its inhibitory effect on insulin secretion.

EphAs modulate activity of Rac1, a Rho-GTPase involved in F-actin remodeling and endocytosis (Murai and Pasquale (2005) Neuron 46, 161). Since Rac1 is also involved in glucose-stimulated insulin secretion (Kowluru and Veluthakal (2005) Diabetes 54, 3523; Li et al. (2004) Am. J. Physiol. Endocrinol. Metab. 286, E818), we investigated whether EphA5-Fc and ephrin-A5-Fc modulated Rac1 activity in MIN6 cells. In line with previous reports (Kowluru and Veluthakal (2005) Diabetes 54, 3523; Li et al. (2004) Am. J. Physiol. Endocrinol. Metab. 286, E818), glucose-stimulation significantly enhanced Rac1 activity (Fc, FIGS. 5F and 5G). Consistent with its stimulatory effects on insulin secretion, we found that EphA5-Fc stimulated Rac1 activity under basal and stimulatory conditions (compare Fc with EphA5-Fc in FIG. 5G). In contrast, ephrin-A5-Fc inhibited Rac1 activity in confluent MIN6 cells under stimulatory conditions, consistent with its inhibitory effect on insulin secretion (compare Fc with ephrin-A5-Fc in FIG. 5G). In addition, EphA5-Fc and ephrin-A5-Fc required Rac1 activity for their effects on glucose-stimulated insulin secretion (compare DN-Rac1 with empty vector in FIG. 5H). These results suggest that EphA5-Fc and ephrin-A5-Fc modulate glucose-stimulated insulin secretion by affecting Rac1 activity and F-actin in β-cells.

Since Eph-ephrin signaling was also shown to affect connexin localization and gap junction communication (Mellitzer et al. (1999) Nature 400: 77; Davy et al. (2006) PLOS Biol. 4: 1763), we investigated whether connexin-36, the gap junction protein of β-cells (Ravier et al. (2005) Diabetes 54: 1798), was required for the effects of Eph-ephrin signaling on insulin secretion (FIG. 13). We showed that the stimulatory effect of EphA5-Fc on basal and glucose-stimulated insulin secretion required connexin-36 (compare Fc with EphA5-Fc in FIG. 13C), whereas the inhibitory effect of ephrin-A5-Fc did not (compare Fc with ephrin-A5-Fc in FIG. 13F).

Taken together, these results show that EphA-ephrin-A signaling affects several β-cell components required for regulating insulin secretion: F-actin, Rac1 and connexin-36.

### EXAMPLE 9

### Glucose-induced Dephosphorylation of EphA5

EphAs are receptor tyrosine kinases (RTKs) that oligomerize upon ephrin-A binding (Kullander and Klein (2002) Nat. Rev. Mol. Cell Biol. 3, 475). The oligomerization brings the cytoplasmic EphA RTK domains in close proximity to each other, thereby allowing their trans-phosphorylation, which promotes EphA forward signaling. The level of kinase-dependent EphA forward signaling can therefore be determined by measuring the levels of EphA tyrosine phosphorylation. Here we investigated the hypothesis that EphA forward signaling was active at low glucose concentrations in order to suppress insulin secretion (FIG. 6A). We also investigated whether, upon glucose stimulation, EphA forward signaling was downregulated by dephosphorylation (FIG. 6B), thus allowing predominance of ephrin-A reverse signaling, which stimulated insulin secretion.

In support of this hypothesis, we found that the EphA5 phosphorylation levels were higher at a low glucose concentration (2 mM) compared to all depicted time points after glucose stimulation (25 mM) in both islets and MIN6 cells (FIGS. 6C and 6D). In islets, the presence of phosphorylated EphA5 decreased shortly after glucose stimulation (compare column 1 with columns 2 to 4 in FIG. 6C). However, when the islets were brought back to low glucose concentration, EphA5 phosphorylation reached basal levels again (compare column 1 with column 5 in FIG. 6C). The level of EphA5 phosphorylation was easier to determine in MIN6 cells, because these cells were not mixed with non-β-cells and could be obtained in large quantities (FIG. 6D). As with islets, the amount of phosphorylated EphA5 decreased shortly after glucose stimulation (compare column 1 with columns 2 to 5 in FIG. 6D). At the 5^{th} minute, EphA5 phosphorylation was reduced to about 20% of basal levels (compare column 1 with column 3 in FIG. 6D). Similar to the islets, bringing back glucose-stimulated MIN6 cells to a low glucose concentration fully restored basal EphA5 phosphorylation levels (compare last column with first column in FIG. 6D). To demonstrate that EphA5 was not the only EphA to be dephosphorylated in β-cells, we performed experiments with other members of the EphA family and observed a similar dephosphorylation upon glucose stimulation (data not shown).

Since ephrin-A5-Fc inhibited insulin secretion, we tested whether this effect correlated with the phosphorylation levels of EphA5. We noticed that treatment of islets with ephrin-A5-Fc resulted in elevated EphA5 phosphorylation under stimulatory conditions (compare column 3 with last column in FIG. 6C). In addition, ephrin-A5-Fc treatment of non-confluent MIN6 cells significantly increased EphA5 phosphorylation also under basal conditions (FIGS. 3E and 3F; FIGS. 6E and 6F). Thus, EphA5 phosphorylation levels correlate with inhibition of insulin secretion.

We then tested whether a protein tyrosine phosphatase (PTP) was involved in dephosphorylating EphAs in response to glucose stimulation (FIG. 6B). We observed that 10 μM peroxovanadate (PV), a PTP inhibitor, effectively prevented glucose-induced EphA5 dephosphorylation (FIG. 6G), and even increased EphA5 phosphorylation levels at stimulatory conditions above the levels characteristic of basal conditions (compare columns in FIGS. 6E and 6G). These results therefore suggest that EphA forward signaling is active at low glucose concentrations in order to inhibit insulin secretion (FIG. 6A), and that EphA forward signaling is downregulated by dephosphorylation, upon glucose stimulation (FIG. 6B).

### EXAMPLE 10

### EphA5 Dephosphorylation is Required for Glucose-stimulated Insulin Secretion

It was previously shown that treatment of islets with 10 μM PV strongly reduced glucose-stimulated insulin secretion (Gogg et al. (2001) Biochem. Biophys. Res. Commun. 280, 1161). Based on our experiments, we hypothesized that PV inhibited glucose-stimulated insulin secretion partially by preventing glucose-induced EphA dephosphorylation (FIG. 7A). After confirming that 10 μM PV reduced glucose-stimulated insulin secretion (FIGS. 7D and 7E), we tested whether insulin secretion could be rescued in PV-treated MIN6 cells by overexpression of DN-EphA5 (FIG. 7B). We also tested whether insulin secretion could be further reduced in PV-treated MIN6 cells by overexpression of wt EphA5 (FIG. 7C). We found that DN-EphA5 partially rescued glucose-stimulated insulin secretion in PV-treated MIN6 cells (compare Empty vector/PV with DN-EphA5/PV in FIG. 7E). In contrast, wt EphA5 reduced glucose-stimulated insulin secretion in PV-treated MIN6 cells even further (compare empty vector/PV with wt EphA5/PV in FIG. 7E). We conclude that downregulation of EphA forward signaling is essential for glucose-stimulated insulin secretion.

1. A method of identifying a compound suitable as a lead compound and/or as a medicament for the treatment and/or prevention of diabetes, the method comprising the steps of
(a) contacting a test compound with a cell comprising a protein, wherein said protein
(i) comprises or consists of the amino acid sequence of any one of SEQ ID NOs: 1 to 29;
(ii) is encoded by a nucleic acid molecule comprising or consisting of the sequence of any one of SEQ ID NOs: 59 to 87;
(iii) is a fragment of the protein according to (i) or (ii) and exhibits Eph-Receptor-Tyrosine-Kinase activity; or
(iv) has a sequence at least 75% identical with the protein according to (i) or (ii) or with the fragment according to (iii) and exhibits Eph-Receptor-Tyrosine-Kinase activity; and
(b) determining whether said test compound, upon contacting in step (a) inhibits the Eph receptor tyrosine kinase activity of said protein wherein said inhibition indicates a compound suitable as a lead compound and/or as a medicament for the treatment and/or prevention of diabetes.

2. A method of identifying a compound suitable as a lead compound and/or as a medicament for the treatment and/or prevention of diabetes, the method comprising the steps of
(a) contacting a test compound with a protein, wherein said protein
(i) comprises or consists of the amino acid sequence of any one of SEQ ID NOs: 1 to 29, or
(ii) is encoded by a nucleic acid molecule comprising or consisting of the sequence of any one of SEQ ID NOs: 59 to 87, or
(iii) is a fragment of the protein according to (i) or (ii) and exhibits Eph-Receptor-Tyrosine-Kinase activity, or
(iv) has a sequence at least 75% identical with the protein according to (i) or (ii) or with the fragment according to (iii) and exhibits Eph-Receptor-Tyrosine-Kinase activity; and
(b) determining whether said test compound, upon contacting in step (a) inhibits the Eph receptor tyrosine kinase activity of said protein wherein said inhibition indicates a compound suitable as a lead compound and/or as a medicament for the treatment and/or prevention of diabetes.

3. A method of identifying a compound suitable as a lead compound and/or as a medicament for the treatment and/or prevention of diabetes, the method comprising the steps of
(a) contacting a test compound with a cell comprising a protein, wherein said protein
(i) comprises or consists of the amino acid sequence of any one of SEQ ID NOs: 48to58,or
(ii) is encoded by a nucleic acid molecule comprising or consisting of the sequence of any one of SEQ ID NOs: 106 to 116, or
(iii) is a fragment of the protein according to (i) or (ii) and acts as a protein tyrosine phosphatase for Eph receptor tyrosine kinases; or
(iv) has a sequence at least 75% identical with the protein according to (i) or (ii) or with the fragment according to (iii) and acts as a protein tyrosine phosphatase for Eph receptor tyrosine kinases; and
(b) determining whether said test compound, upon contacting in step (a) activates the expression and/or activity of said protein wherein said activation indicates a compound suitable as a lead compound and/or as a medicament for the treatment and/or prevention of diabetes.

4. A method of identifying a compound suitable as a lead compound and/or as a medicament for the treatment and/or prevention of diabetes, the method comprising the steps of
(a) contacting a test compound with a protein, wherein said protein
(i) comprises or consists of the amino acid sequence of any one of SEQ ID NOs: 48 to 58, or
(ii) is encoded by a nucleic acid molecule comprising or consisting of the sequence of any one of SEQ ID NOs: 106 to 116, or
(iii) is a fragment of the protein according to (i) or (ii) and acts as a protein tyrosine phosphatase for Eph receptor tyrosine kinases; or
(iv) has a sequence at least 75% identical with the protein according to (i) or (ii) or with the fragment according to (iii) and acts as a protein tyrosine phosphatase for Eph receptor tyrosine kinases; and
(b) determining whether said test compound, upon contacting in step (a) enhances the activity of said protein wherein said enhancement indicates a compound suitable as a lead compound and/or as a medicament for the treatment and/or prevention of diabetes.

5. A method of identifying a compound suitable as a lead compound and/or as a medicament for the treatment and/or prevention of diabetes, the method comprising the steps of
(a) contacting a test compound with a cell comprising a protein, wherein said protein
(i) comprises or consists of the amino acid sequence of any one of SEQ ID NOs: 30 to 47, or
(ii) is encoded by a nucleic acid molecule comprising or consisting of the sequence of any one of SEQ ID NOs: 88 to 105, or
(iii) is a fragment of the protein according to (i) or (ii) and exhibits ephrin activity, or
(iv) has a sequence at least 75% identical with the protein according to (i) or (ii) or with the fragment according to (iii) and exhibits ephrin activity; and
(b) determining whether said test compound, upon contacting in step (a) activates the expression and/or activity of said protein wherein said activation indicates a compound suitable as a lead compound and/or as a medicament for the treatment and/or prevention of diabetes.

6. The method of any one of claims 1 to 5, wherein said cell is a pancreatic beta-cell or a cell from a beta-cell line.

7. The method of any one of claims 1 to 5, wherein determining in step (b) comprises quantifying insulin secretion.

8. The method of any one of claims 1 to 5 wherein determining in step (b) comprises quantifying F-actin in said cell, wherein the quantity of F-actin is determined at stimulatory glucose concentrations and wherein a decreased quantity of F-actin in the cell contacted with the test compound indicates a compound suitable as a lead compound and/or as a medicament for the treatment and/or prevention of diabetes.

9. The method of any one of claims 1 to 5, wherein said protein is comprised in a cell expressing, or transfected with, a nucleic acid encoding said protein.

10. The method of any one of claims 1 to 5, wherein said cell is comprised in a non-human animal.

11. The method of claim 10, wherein determining in step (b) involves quantifying insulin secretion.
